SEQUENCE LISTING (22 sequences)

SEQ ID NO: 1 - protein, 4 aa, synthetic construct
  REGION 1..4: note = peptide
KRSS

SEQ ID NO: 2 - protein, 5 aa, synthetic construct
  REGION 1..5: note = peptide
PGESS

SEQ ID NO: 3 - protein, 4 aa, synthetic construct
  REGION 1..4: note = peptide
SLTQ

SEQ ID NO: 4 - protein, 4 aa, synthetic construct
  REGION 1..4: note = peptide
AMGN

SEQ ID NO: 5 - protein, 5 aa, synthetic construct
  REGION 1..5: note = peptide
GDRLV

SEQ ID NO: 6 - protein, 7 aa, synthetic construct
  REGION 1..7: note = peptide
RGGKRSS

SEQ ID NO: 7 - protein, 7 aa, synthetic construct
  REGION 1..7: note = peptide
SLTQDQG

SEQ ID NO: 8 - protein, 7 aa, synthetic construct
  REGION 1..7: note = peptide
ADAQADV

SEQ ID NO: 9 - protein, 7 aa, synthetic construct
  REGION 1..7: note = peptide
RAKGRDA

SEQ ID NO: 10 - protein, 7 aa, synthetic construct
  REGION 1..7: note = peptide
KASRLGR

SEQ ID NO: 11 - protein, 7 aa, synthetic construct
  REGION 1..7: note = peptide
RGASMDG

SEQ ID NO: 12 - protein, 7 aa, synthetic construct
  REGION 1..7: note = peptide
GRPGESS

SEQ ID NO: 13 - protein, 7 aa, synthetic construct
  REGION 1..7: note = peptide
SRTEGDV

SEQ ID NO: 14 - protein, 9 aa, synthetic construct
  REGION 1..9: note = peptide
CRGGKRSSC

SEQ ID NO: 15 - protein, 9 aa, synthetic construct
  REGION 1..9: note = peptide
CSLTQDQGC

SEQ ID NO: 16 - protein, 9 aa, synthetic construct
  REGION 1..9: note = peptide
CADAQADVC

SEQ ID NO: 17 - protein, 9 aa, synthetic construct
  REGION 1..9: note = peptide
CRAKGRDAC

SEQ ID NO: 18 - protein, 9 aa, synthetic construct
  REGION 1..9: note = peptide
CKASRLGRC

SEQ ID NO: 19 - protein, 9 aa, synthetic construct
  REGION 1..9: note = peptide
CRGASMDGC

SEQ ID NO: 20 - protein, 9 aa, synthetic construct
  REGION 1..9: note = peptide
CGRPGESSC

SEQ ID NO: 21 - protein, 9 aa, synthetic construct
  REGION 1..9: note = peptide
CSRTEGDVC

SEQ ID NO: 22 - protein, 9 aa, synthetic construct
  REGION 1..9: note = peptide
CSGRRGKSC

(19) United States
(12) Patent Application Publication
(10) Pub. No.: US 2024/0252579 A1
(43) Pub. Date: Aug. 1, 2024

(54) USE OF CNS-HOMING TARGETING PEPTIDES FOR TREATING NEUROLOGICAL DISORDERS

(71) Applicant: SILO PHARMA, INC., Englewood Cliffs, NJ (US)

(72) Inventor: Eric Weisblum, Englewood Cliffs, NJ (US)

(21) Appl. No.: 18/423,557

(22) Filed: Jan. 26, 2024

Related U.S. Application Data
(60) Provisional application No. 63/482,192, filed on Jan. 30, 2023.

(51) Int. Cl. A61K 38/04; A61K 45/06

(52) U.S. Cl. CPC A61K 38/04; A61K 45/06

(57) ABSTRACT

Disclosed herein include methods, compositions, and kits suitable for use in treating, prevent, delaying, or reversing neurological disorders, including dementia and Alzheimer's disease. In some embodiments, the method comprises administering to a subject in a need thereof a therapeutically effective amount of a composition comprising a central nervous system (CNS) homing peptide.

### CROSS-REFERENCE TO RELATED APPLICATIONS

This application claims the benefit under 35 U.S.C. § 119(e) of U.S. Provisional Patent Application Ser. No. 63/482,192, filed Jan. 30, 2023, the content of this related application is incorporated herein by reference in its entirety for all purposes.

### REFERENCE TO SEQUENCE LISTING

The present application is being filed along with a Sequence Listing in electronic format. The Sequence Listing is provided as a file entitled 72GJ-325708-P_SequenceListing, created Jan. 25, 2023, which is 27 kilobytes in size. The information in the electronic format of the Sequence Listing is incorporated herein by reference in its entirety.

### BACKGROUND

### Field

The present disclosure relates generally to the fields of neurobiology, molecular biology and medicine. One aspect relates to the treatment and prevention of neurological disorders, for example dementia, Alzheimer's disease and related disorders (e.g., Alzheimer's disease-related dementia) with a central nervous system homing peptide.

### Description of the Related Art

Dementia is defined by chronic, acquired loss of one, two or more cognitive abilities caused by brain disease or injury. The most common cause of dementia is Alzheimer's disease (AD), accounting for 60 to 80 percent of cases. AD is a progressive neurodegenerative disorder that is associated with the destruction of higher brain structures, such as those involved in memory and cognition. The disease leads to deficits in cognitive function and declines in memory, learning, language, and in the ability to perform intentional and purposeful movements. AD is also accompanied by concomitant behavioral, emotional, interpersonal, and social deterioration. Histopathologically, AD can be characterized by the accumulation of amyloid plaques comprising the amyloid-β (Aβ) peptide and neurofibrillary tangles (NFTs) made of the tau protein. Under normal conditions, the soluble Aβ peptide is produced and secreted by neurons and subsequently cleared from the brain via cerebral spinal fluid (CSF) pathways. However, in subjects with AD, the Aβ peptide appears to aggregate into higher-order species to form soluble oligomers and insoluble plaques in a concentration-dependent manner. This aggregation may initiate many neurotoxic events including disrupted brain metabolism, neuroinflammation, reduced functional connectivity, synaptic and neuronal loss, and/or formation of NFTs.

AD currently has no cure, and treatment options do not inhibit the pathological progression of AD, are mainly palliative, and/or may have multiple, troubling side effects. For example, preventative and/or therapeutic strategies targeting the Aβ peptide and/or its precursors (e.g., Aβ immunotherapy and inhibition of β- and γ-secretases) have been toxic and/or ineffective at reducing AD pathology in clinical trials. Clinical trials involving amyloid beta vaccines (e.g., bapineuzumab) have failed due to lack of cognitive benefit. Gamma-secretase inhibitors (e.g., semagacestat) have failed clinical trials for worsening of cognitive deficits in subjects. Even existing medications like acetylcholinesterase inhibitors (e.g., donepezil and rivastigmine) and N-methyl-D-aspartate (NMDA)-receptor antagonists (e.g., memantine) demonstrate only mild cognitive benefits. There is an urgent need for effective treatments for dementia and AD. Compositions and methods for delaying or reducing the likelihood of onset of dementia and Alzheimer's disease are also urgently needed.

### SUMMARY

Disclosed herein include methods of treating, preventing, or reversing dementia in a subject in need thereof. In some embodiments, the method comprises administering to the subject a therapeutically effective amount of a composition comprising a central nervous system (CNS) homing peptide, thereby treating, preventing, or reversing dementia in the subject. In some embodiments, the composition does not comprise any additional therapeutic agent. In some embodiments, the dementia is AD-related dementia, vascular dementia, Lewy body dementia, fronto-temporal dementia, or mixed dementia. In some embodiments, the subject is not receiving any additional therapy for treating, preventing, or reversing dementia. In some embodiments, the additional therapy is discontinued prior to administration of the composition. In some embodiments, the subject is not being administered any additional therapeutic agent for treating, preventing, or reversing dementia.

Disclosed herein include methods of treating AD, or delaying or reducing the likelihood of onset of AD. In some embodiments, the method comprises administering to a subject in need thereof a therapeutically effective amount of a composition comprising a CNS homing peptide, thereby treating AD or delaying or reducing the likelihood of onset of AD in the subject. In some embodiments, the composition does not comprise any additional therapeutic agent. In some embodiments, the subject is not receiving any additional therapy for treating AD or delaying or reducing the likelihood of onset of AD. In some embodiments, the additional therapy is discontinued prior to administration of the composition. In some embodiments, the subject is not being administered any additional therapeutic agent for treating AD or delaying or reducing the likelihood of onset of AD.

Disclosed herein include methods of treating, preventing, or reversing cognitive decline in clinical or pre-clinical AD. In some embodiments, the method comprises administering to a subject in need thereof a therapeutically effective amount of a composition comprising a CNS homing peptide, thereby treating, preventing, or reversing cognitive decline in clinical or pre-clinical AD in the subject. In some embodiments, the composition does not comprise any additional therapeutic agent. In some embodiments, the subject is not receiving any additional therapy for treating, preventing, or reversing cognitive decline in clinical or pre-clinical AD. In some embodiments, the additional therapy is discontinued prior to administration of the composition. In some embodiments, the subject is not being administered any additional therapeutic agent for treating, preventing, or reversing cognitive decline in clinical or pre-clinical AD.

Disclosed herein include methods of delaying or reversing the progression of AD. In some embodiments, the method comprises administering to a subject in need thereof a therapeutically effective amount of a composition comprising a central nervous system (CNS) homing peptide, thereby delaying or reversing the progression of AD in the subject. In some embodiments, the composition does not comprise any additional therapeutic agent. In some embodiments, the subject is not receiving any additional therapy for delaying or reversing the progression of AD. In some embodiments, the additional therapy is discontinued prior to administration of the composition. In some embodiments, the subject is not being administered any additional therapeutic agent for delaying or reversing the progression of AD.

In some embodiments, the CNS homing peptide comprises an amino acid sequence of any one of SEQ ID NOs: 1-22. In some embodiments, the CNS homing peptide is capable of homing to the entorhinal cortex, the cerebral cortex, the hippocampus, or any combination thereof.

In some embodiments, the subject is a mammal. In some embodiments, the subject is a human. In some embodiments, the human is over 40, over 60, or over 70 years old. In some embodiments, the subject in need thereof is suspected of having Alzheimer's disease.

The method can comprise identifying the subject in need thereof, wherein the subject in need thereof is a subject at a risk of having AD, a subject having AD, or a subject suspected of having AD. The method can comprise identifying the subject in need thereof as a subject having mild cognitive impairment or dementia associated with AD, early stage AD, mid-stage AD, late-stage AD, sporadic AD, or familial AD. In some embodiments, the subject in need thereof has AD or is at a risk of developing AD. In some embodiments, the subject is diagnosed with mild cognitive impairment associated with AD, early stage AD, mid-stage AD, or late-stage AD. In some embodiments, the subject is diagnosed with AD-related dementia. In some embodiments, the subject in need thereof has at least one mutation in one or more genes associated with Alzheimer's disease. In some embodiments, the one or more genes comprise apolipoprotein E (APOE), amyloid precursor protein (APP), presenilin 1 (PSEN1), and/or presenilin 2 (PSEN2). In some embodiments, the AD is sporadic Alzheimer's disease. In some embodiments, the AD is familial AD.

In some embodiments, the composition is a pharmaceutical composition comprising the CNS homing peptide and at least one pharmaceutically acceptable excipient. In some embodiments, the composition is administered to the subject by intravenous administration. In some embodiments, the composition is formulated for intravenous administration. In some embodiments, the composition is administered intravenously as a bolus, injection, infusion, or prolonged infusion. In some embodiments, the composition is administered to the subject by subcutaneous injection, nasal administration, pulmonary administration, oral administration, parenteral administration, or nebulization. In some embodiments, the composition is administered to the subject once, twice, or three times a day. In some embodiments, the composition is administered to the subject daily, every two days, every three days, every week, every two weeks, or every month.

In some embodiments, the additional therapeutic agent comprises: (i) an acetylcholinesterase inhibitor selected from the group consisting of donepezil hydrochloride (ARICEPT, MEMAC), physostigmine salicylate (ANTILIRIUM), physostigmine sulfate (ESERINE), metrifonate, neostigmine, ganstigmine, pyridostigmine (MESTINON), ambenonium (MYTELASE), demarcarium, Debio 9902, rivastigmine (EXELON), ladostigil, NP-0361, galantamine hydrobromide (RAZADYNE, RIMINYL, NIVALIN), tacrine (COGNEX), tolserine, velnacrine maleate, memoquin, huperzine A (HUP-A), phenserine, edrophonium (ENLON, TENSILON), and INM-176; (ii) an amyloid-β (or fragments thereof) selected from the group consisting of A1-15 conjugated to pan HLA DR-binding epitope (PADRE), ACC-001, ACI-01, ACI-24, AN-1792, Affitope AD-01, CAD106, and V-950; (iii) an antibody to amyloid-β (or fragments thereof) selected from the group consisting of lecanemab (LEQEMBI), aducanumab (ADUHELM), ponezumab, solanezumab, bapineuzumab, AAB-002, ACI-01-Ab7, BAN-2401, intravenous Ig (GAMMAGARD), LY2062430 (humanized m266), R1450, ACU-5A5, and huC091; (iv) an amyloid-lowering or -inhibiting agent (including those that reduce amyloid production, accumulation and fibrillization) selected from the group consisting of dimebon, davunetide, eprodisate, leuprolide, SK-PC-B70M, celecoxib, lovastatin, anapsos, oxiracetam, pramiracetam, varenicline, nicergoline, colostrinin, bisnorcymserine, NIC5-15 (Humanetics), E-2012 (Eisai), pioglitazone, clioquinol, PBT2 (Prana Biotechnology), flurbiprofen (ANSAID, FROBEN) and its R-enantiomertarenflurbil (FLURIZAN), nitroflurbiprofen, fenoprofen (FENOPRON, NALFON), ibuprofen (ADVIL, MOTRIN, NUROFEN), ibuprofen lysinate, meclofenamic acid, meclofenamate sodium (MECLOMEN), indomethacin (INDOCIN), diclofenac sodium (VOLTAREN), diclofenac potassium, sulindac (CLINORIL), sulindac sulfide, diflunisal (DOLOBID), naproxen (NAPROSYN), naproxen sodium (ANAPROX, ALEVE), ARC031, CAD-106 (Cytos), LY450139, insulin-degrading enzyme, the gingko biloba extract EGb-761 (ROKAN, TEBONIN), tramiprosate (CEREBRIL, ALZHEMED), eprodisate (FIBRILLEX, KIACTA), compound W [3,5-bis(4-nitrophenoxy)benzoic acid], NGX-96992, neprilysin, scyllo-inositol, atorvastatin (LIPITOR), simvastatin (ZOCOR), KLVFF-(EEX)3, SKF-74652, ibutamoren mesylate, BACE inhibitors such as ASP-1702, SCH-745966, JNJ-715754, AMG-0683, AZ-12304146, BMS-782450, GSK-188909, NB-533, E2609 and TTP-854; gamma secretase modulators; and RAGE (receptor for advanced glycation end-products) inhibitors, and PTI-777; (v) an alpha-adrenergic receptor agonist selected from the group consisting of guanfacine (INTUNIV, TENEX), clonidine (CATAPRES), metaraminol (ARAMINE), methyldopa (ALDOMET, DOPAMET, NOVOMEDOPA), tizanidine (ZANAFLEX), phenylephrine, methoxamine, cirazoline, guanfacine (INTUNIV), lofexidine, xylazine, modafinil (PROVIGIL), adrafinil, and armodafinil (NUVIGIL); (vi) a beta-adrenergic receptor blocking agent (beta blocker) selected from the group consisting of carteolol, esmolol (BREVIBLOC), labetalol (NORMODYNE, TRANDATE), oxprenolol (LARACOR, TRASACOR), pindolol (VISKEN), propanolol (INDERAL), sotalol (BETAPACE, SOTALEX, SOTACOR), timolol (BLOCADREN, TIMOPTIC), acebutolol (SECTRAL, PRENT), nadolol (CORGARD), metoprolol tartrate (LOPRESSOR), metoprolol succinate (TOPROL-XL), atenolol (TENORMIN), butoxamine, and SR 59230A; (vii) an anticholinergic selected from the group consisting of amitriptyline (ELAVIL, ENDEP), butriptyline, benztropine mesylate (COGENTIN), trihexyphenidyl (ARTANE), diphenhydramine (BENADRYL), orphenadrine (NORFLEX), hyoscyamine, atropine (ATROPEN), scopolamine (TRANSDERM-SCOP), scopolamine methylbromide (PARMINE), dicycloverine (BENTYL, BYCLOMINE, DIBENT, DILOMINE), tolterodine (DETROL), oxybutynin (DITROPAN, LYRINEL XL, OXYTROL), penthienate bromide, propantheline (PRO-BANTHINE), cyclizine, imipramine hydrochloride (TOFRANIL), imipramine maleate (SURMONTIL), lofepramine, desipramine (NORPRAMIN), doxepin (SINEQUAN, ZONALON), trimipramine (SURMONTIL), and glycopyrrolate (ROBINUL); (viii) an anticonvulsant selected from the group consisting of carbamazepine (TEGRETOL, CARBATROL), oxcarbazepine (TRILEPTAL), phenytoin sodium (PHENYTEK), fosphenytoin (CEREBYX, PRODILANTIN), divalproex sodium (DEPAKOTE), gabapentin (NEURONTIN), pregabalin (LYRICA), topirimate (TOPAMAX), valproic acid (DEPAKENE), valproate sodium (DEPACON), 1-benzyl-5-bromouracil, progabide, beclamide, zonisamide (TRERIEF, EXCEGRAN), CP-465022, retigabine, talampanel, and primidone (MYSOLINE); (ix) an antipsychotic selected from the group consisting of lurasidone (LATUDA), aripiprazole (ABILIFY), chlorpromazine (THORAZINE), haloperidol (HALDOL), iloperidone (FANAPTA), flupentixol decanoate (DEPIXOL, FLUANXOL), reserpine (SERPLAN), pimozide (ORAP), fluphenazine decanoate, fluphenazine hydrochloride, prochlorperazine (COMPRO), asenapine (SAPHRIS), Ioxapine (LOXITANE), molindone (MOBAN), perphenazine, thioridazine, thiothixine, trifluoperazine (STELAZINE), ramelteon, clozapine (CLOZARIL), norclozapine (ACP-104), risperidone (RISPERDAL), paliperidone (INVEGA), melperone, olanzapine (ZYPREXA), quetiapine (SEROQUEL), talnetant, amisulpride, ziprasidone (GEODON), blonanserin (LONASEN), and ACP-103; (x) a calcium channel blocker selected from the group consisting of omerizine, ziconotide, nilvadipine (ESCOR, NIVADIL), diperdipine, amlodipine (NORVASC, ISTIN, AMLODIN), felodipine (PLENDIL), nicardipine (CARDENE), nifedipine (ADALAT, PROCARDIA), MEM 1003 and its parent compound nimodipine (NIMOTOP), nisoldipine (SULAR), nitrendipine, lacidipine (LACIPIL, MOTENS), lercanidipine (ZANIDIP), lifarizine, diltiazem (CARDIZEM), verapamil (CALAN, VERELAN), AR-R 18565 (AstraZeneca), and enecadin; (xi) a catechol O-methyltransferase (COMT) inhibitor selected from the group consisting of nitecapone, tolcapone (TASMAR), entacapone (COMTAN), and tropolone; (xii) a central nervous system stimulant selected from the group consisting of atomoxetine, reboxetine, yohimbine, caffeine, phenmetrazine, phendimetrazine, pemoline, fencamfamine (GLUCOENERGAN, REACTIVAN), fenethylline (CAPTAGON), pipradol (MERETRAN), deanol, methylphenidate (DAYTRANA), methylphenidate hydrochloride (RITALIN), dexmethylphenidate (FOCALIN), amphetamine (alone or in combination with other CNS stimulants, e.g., ADDERALL (amphetamine aspartate, amphetamine sulfate, dextroamphetamine saccharate, and dextroamphetamine sulfate)), dextroamphetamine sulfate (DEXEDRINE, DEXTROSTAT), methamphetamine (DESOXYN), lisdexamfetamine (VYVANSE), and benzphetamine (DIDREX); (xiii) a corticosteroid selected from prednisone (STERAPRED, DELTASONE), prednisolone (PRELONE), predisolone acetate (OMNIPRED, PRED MILD, PRED FORTE), prednisolone sodium phosphate (ORAPRED ODT), methylprednisolone (MEDROL), methylprednisolone acetate (DEPO-MEDROL), and methylprednisolone sodium succinate (A-METHAPRED, SOLU-MEDROL); (xiv) a dopamine receptor agonist selected from the group consisting of apomorphine (APOKYN), bromocriptine (PARLODEL), cabergoline (DOSTINEX), dihydrexidine, dihydroergocryptine, fenoldopam (CORLOPAM), lisuride (DOPERGIN), terguride spergolide (PERMAX), piribedil (TRIVASTAL, TRASTAL), pramipexole (MIRAPEX), quinpirole, ropinirole (REQUIP), rotigotine (NEUPRO), SKF-82958, cariprazine, pardoprunox, and sarizotan; (xv) a dopamine receptor antagonist selected from the group consisting of chlorpromazine, fluphenazine, haloperidol, loxapine, risperidone, thioridazine, thiothixene, trifluoperazine, tetrabenazine (NITOMAN, XENAZINE), 7-hydroxyamoxapine, droperidol (INAPSINE, DRIDOL, DROPLETAN), domperidone (MOTILIUM), L-741742, L-745870, raclopride, SB-277011A, SCH-23390, ecopipam, SKF-83566, and metoclopramide (REGLAN); (xvi) a dopamine reuptake inhibitor selected from the group consisting of bupropion, safinamide, nomifensine maleate (MERITAL), vanoxerine and its decanoate ester DBL-583, and amineptine; (xvii) a gamma-amino-butyric acid (GABA) receptor agonist selected from the group consisting of baclofen (LIORESAL, KEMSTRO), siclofen, pentobarbital (NEMBUTAL), progabide (GABRENE), and clomethiazole; (xviii) a histamine 3 (H3) antagonist; (xix) an immunomodulator selected from the group consisting of glatiramer acetate, MBP-8298 (synthetic myelin basic protein peptide), dimethyl fumarate, fingolimod, roquinimex (LINOMIDE), laquinimod, ABT-874 (human anti-IL-12 antibody; Abbott), rituximab (RITUXAN), alemtuzumab (CAMPATH), daclizumab (ZENAPAX), and natalizumab (TYSABRI); (xx) an immunosuppressant selected from the group consisting of methotrexate (TREXALL, RHEUMATREX), mitoxantrone (NOVANTRONE), mycophenolate mofetil (CELLCEPT), mycophenolate sodium (MYFORTIC), azathioprine (AZASAN, IMURAN), mercaptopurine (PURI-NETHOL), cyclophosphamide (NEOSAR, CYTOXAN), chlorambucil (LEUKERAN), cladribine (LEUSTATIN, MYLINAX), alpha-fetoprotein, etanercept (ENBREL), and 4-(benzyloxy)-5-[(5-undecyl-2H-pyrrol-2-ylidene)methyl]-1H,1′H-2,2′-bipyrrole; (xxi) an interferon selected from the group consisting of interferon beta-la (AVONEX, REBIF) and interferon beta-1b (BETASERON, BETAFERON);

(xxii) a levodopa (or its methyl or ethyl ester), alone or in combination with a DOPA decarboxylase inhibitor (e.g., carbidopa (SINEMET, CARBILEV, PARCOPA), benserazide (MADOPAR), α-methyldopa, monofluromethyldopa, difluoromethyldopa, brocresine, or m-hydroxybenzylhydrazine); (xxiii) a N-methyl-D-aspartate (NMDA) receptor antagonist selected from the group consisting of memantine (NAMENDA, AXURA, EBIXA), amantadine (SYMMETREL), acamprosate (CAMPRAL), besonprodil, ketamine (KETALAR), delucemine, dexanabinol, dexefaroxan, dextromethorphan, dextrorphan, traxoprodil, CP-283097, himantane, idantadol, ipenoxazone, L-701252, lancicemine, levorphanol (DROMORAN), LY-233536, LY-235959, methadone, (DOLOPHINE), neramexane, perzinfotel, phencyclidine, tianeptine (STABLON), dizocilpine, EAB-318, ibogaine, voacangine, tiletamine, riluzole (RILUTEK), aptiganel (CERESOTAT), gavestinel, and remacimide; (xxiv) a monoamine oxidase (MAO) inhibitor selected from the group consisting of selegiline (EMSAM), selegiline hydrochloride (L-deprenyl, ELDEPRYL, ZELAPAR), dimethylselegilene, brofaromine, phenelzine (NARDIL), tranylcypromine (PARNATE), moclobemide (AURORIX, MANERIX), befloxatone, safinamide, isocarboxazid (MARPLAN), nialamide (NIAMID), rasagiline (AZILECT), iproniazide (MARSILID, IPROZID, IPRONID), CHF-3381 (Chiesi Farmaceutici), iproclozide, toloxatone (HUMORYL, PERENUM), bifemelane, desoxypeganine, harmine, harmaline, linezolid (ZYVOX, ZYVOXID), and pargyline (EUDATIN, SUPIRDYL); (XXv) a muscarinic receptor agonist selected from the group consisting of cevimeline, levetiracetam, bethanechol chloride (DUVOID, URECHOLINE), itameline, pilocarpine (SALAGEN), NGX267, arecoline, L-687306, L-689660, furtrethonium iodide (FURAMON, FURANOL), furtrethonium benzensulfonate, furtrethonium p-toluenesulfonate, McN-A-343, oxotremorine, sabcomeline, AC-90222, and carbachol (CARBASTAT, MIOSTAT, CARBOPTIC); (xxvi) a neuroprotective drug selected from the group consisting of bosutinib, condoliase, airmoclomol, lamotrigine, perampanel, aniracetam, minaprime, riluzole, N-hydroxy-1,2,4,9-tetrahydro-3H-carbazol-3-imine, desmoteplase, anatibant, astaxanthin, neuropeptide NAP, neurostrol, perampenel, ispronicline, bis(4-β-D-glucopyranosyloxybenzyl)-2-β-D-glucopyranosyl-2-isobutyltartrate (also known as dactylorhin B or DHB), formobactin, xaliproden (XAPRILA), lactacystin, dimeboline hydrochloride (DIMEBON), disufenton (CEROVIVE), arundic acid (ONO-2506, PROGLIA, CEREACT), citicoline, edaravone (RADICUT), AEOL-10113, AEOL-10150, AGY-94806, granulocyte-colony stimulating factor, BAY-38-7271, ancrod (VIPRINEX, ARWIN), DP-b99, HF-0220 (17-ß-hydroxyepiandrosterone; Newron Pharmaceuticals), HF-0420 (also known as oligotropin), pyridoxal 5′-phosphate, microplasmin, S-18986, piclozotan, NPO31112, tacrolimus, L-seryl-L-methionyl-L-alanyl-L-lysyl-L-glutamyl-glycyl-L-valine, AC-184897, ADNF-14, stilbazulenyl nitrone, SUN-N8075, and zonampanel; (xxvii) a nicotinic receptor agonist selected from the group consisting of epibatidine, bupropion, CP-601927, varenicline, ABT-089, ABT-594, AZD-0328, EVP-6124, R3487, TC-4959 and TC-5619 (both Targacept), and RJR-2403; (xxviii) a norepinephrine (noradrenaline) reuptake inhibitor selected from the group consisting of atomoxetine (STRATTERA), doxepin (APONAL, ADAPIN, SINEQUAN), nortriptyline (AVENTYL, PAMELOR, NORTRILEN), amoxapine (ASENDIN, DEMOLOX, MOXIDIL), reboxetine (EDRONAX, VESTRA), viloxazine (VIVALAN), maprotiline (DEPRILEPT, LUDIOMIL, PSYMION), bupropion (WELLBUTRIN), and radaxafine; (xxix) a phosphodiesterase (PDE) inhibitor; (xxx) a quinoline selected from the group consisting of quinine (including its hydrochloride, dihydrochloride, sulfate, bisulfate and gluconate salts), chloroquine, sontoquine, hydroxychloroquine (PLAQUENIL), mefloquine (LARIAM), and amodiaquine (CAMOQUIN, FLAVOQUINE); (xxxi) a β-secretase inhibitor selected from the group consisting of ASP-1702, SCH-745966, JNJ-715754, AMG-0683, AZ-12304146, BMS-782450, GSK-188909, NB-533, LY-2886721, E-2609, HPP-854, (+)-phenserine tartrate (POSIPHEN), LSN-2434074, KMI-574, SCH-745966, Ac-rER (N2-acetyl-D-arginyl-L-arginine), Ioxistatin, and CA074Me; (xxxii) a y-secretase inhibitor and/or modulator selected from the group consisting of BMS-708163 (Avagacest), DSP8658 (Dainippon), ITI-009, L-685458, ELAN-G, ELAN-Z, 4-chloro-N-[(2S)-3-ethyl-1-hydroxypentan-2-yl]benzenesulfonamide; (xxxiii) a serotonin (5-hydroxytryptamine) 1A (5-HTIA) receptor antagonist selected from the group consisting of spiperone, levo-pindolol, BMY 7378, NAD-299, S-(−)-UH-301, NAN 190, lecozotan; (xxxiv) a serotonin (5-hydroxytryptamine) 2C (5-HT2c) receptor agonist selected from the group consisting of vabicaserin and zicronapine; (xxxv) a serotonin (5-hydroxytryptamine) 4 (5-HT4) receptor agonist; (xxxvi) a serotonin (5-hydroxytryptamine) 6 (5-HT6) receptor antagonist selected from the group consisting of A-964324, AVI-101, AVN-211, mianserin (TORVOL, BOLVIDON, NORVAL), methiothepin, ritanserin, ALX-1161, ALX-1175, MS-245, LY-483518, MS-245, Ro 04-6790, Ro 43-68544, Ro 63-0563, Ro 65-7199, Ro 65-7674, SB-399885, SB-214111, SB-258510, SB-271046, SB-357134, SB-699929, SB-271046, SB-742457, Lu AE58054, and PRX-07034 (Epix); (xxxvii) a serotonin (5-HT) reuptake inhibitor selected from the group consisting of alaproclate, citalopram (CELEXA, CIPRAMIL), escitalopram (LEXAPRO, CIPRALEX), clomipramine (ANAFRANIL), duloxetine (CYMBALTA), femoxetine (MALEXIL), fenfluramine (PONDIMIN), norfenfluramine, fluoxetine (PROZAC), fluvoxamine (LUVOX), indalpine, milnacipran (IXEL), paroxetine (PAXIL, SEROXAT), sertraline (ZOLOFT, LUSTRAL), trazodone (DESYREL, MOLIPAXIN), venlafaxine (EFFEXOR), zimelidine (NORMUD, ZELMID), bicifadine, desvenlafaxine (PRISTIQ), brasofensine, vilazodone, cariprazine, neuralstem, and tesofensine; (xxxviii) a trophic factor selected from the group consisting of nerve growth factor (NGF), basic fibroblast growth factor (bFGF; ERSOFERMIN), neurotrophin-3 (NT-3), cardiotrophin-1, brain-derived neurotrophic factor (BDNF), neublastin, meteorin, and glial-derived neurotrophic factor (GDNF), and agents that stimulate production of trophic factors, such as propentofylline, idebenone, PYM50028 (COGANE; Phytopharm), and AIT-082 (NEOTROFIN); (xxxix) a Glycine transporter-1 inhibitor selected from the group consisting of paliflutine, ORG-25935, JNJ-17305600, and ORG-26041; (xl) an AMPA-type glutamate receptor modulator selected from the group consisting of perampanel, mibampator, selurampanel, GSK-729327, N-{(3S,4S)-4-[4-(5-cyanothiophen-2-yl)phenoxy]tetrahydro-furan-3-yl}propane-2-sulfonamide; (xli) a Janus kinase inhibitor (JAK) selected from tofacitinib, ruxolitinib, baricitinib, CYT387, GLPG0634, lestaurtinib, pacritinib, and TG101348; or (xlii) an Interleukin-1 receptor-associated kinase 4 inhibitor (IRAK4).

In some embodiments, administering the composition reduces formation of plaques in the brain of the subject. In some embodiments, administering the composition reduces amyloid fibril formation in the brain of the subject. In some embodiments, administering the composition reduces amyloid-induced cellular toxicity or microglial activation in the brain of the subject. In some embodiments, administering the composition reduces amyloid-induced neurotoxicity in the brain of the subject. In some embodiments, administering the composition reduces the rate or amount of amyloid aggregation, fibril formation, or deposition in the brain of the subject. In some embodiments, administering the composition lessens the degree of amyloid deposition, reduces amyloid-induced inflammation, results in reduction of neuroinflammation, or a combination thereof in the brain of the subject. In some embodiments, administering the composition reduces or slows down the formation of tangles containing hyperphosphorylated tau in the brain of the subject. In some embodiments, administering the composition reduces the concentration or the amount of phosphorylated tau in the brain of the subject.

In some embodiments, the progression or onset of AD is measured quantitatively or qualitatively by at least one technique selected from the group consisting of electroencephalogram (EEG), neuroimaging, functional MRI, structural MRI, diffusion tensor imaging (DTI), [18F]fluorodeoxyglucose (FDG) PET, agents that label amyloid beta or tau, [18F]F-dopa PET, radiotracer imaging, volumetric analysis of regional tissue loss, specific imaging markers of abnormal protein deposition, multimodal imaging, and biomarker analysis (plasma or cerebrospinal fluid). The method can comprise measuring at least one AD symptom in the subject before administering the composition to the subject, after administering the composition to the subject, or both. In some embodiments, administering the composition treats or prevents at least one AD symptom. In some embodiments, administering the composition treats or prevents at least one AD symptom by at least about 10%. In some embodiments, the progression or onset of AD is slowed or reversed by at least about 5%. In some embodiments, the progression or onset of AD is evaluated by measuring at least one AD symptom.

In some embodiments, the AD symptom is selected from the group consisting of: (a) a symptom from the Integrated Alzheimer's Disease Rating Scale (iADRS) selected from the group consisting of personal belonging management, selection of clothes, ability to dress self, ability to clean habitation, financial management ability, writing ability, ability to keep appointments, ability to use telephone, ability to prepare food for self, travel ability, awareness of current events, reading ability, interest in television, ability to shop for self, ability to remain alone, ability to perform chores, ability to perform a hobby or game, driving ability, self-management of medications, ability to initiate and finish complex tasks, and ability to initiate and finish simple tasks; (b) a symptom from the Alzheimer's Disease Assessment Scale-Cognitive subscale (ADAS-Cog) selected from the group consisting of learning, naming, command following, ideational praxis, constructional praxis, orientation, and recognition memory; (c) a symptom from the Alzheimer's Disease Cooperative Study-instrumental Activities of Daily Living (ADCS-iADL) wherein the symptom is any of the symptoms recited in (a) or (b); (d) constipation; (e) depression; (f) cognitive impairment; (g) short term memory impairment; (h) long term memory impairment; (i) concentration impairment; (j) coordination impairment; (k) mobility impairment; (l) speech impairment; (m) mental confusion; (n) sleep problem, sleep disorder, or sleep disturbance; (o) circadian rhythm dysfunction; (p) REM disturbed sleep; (q) REM behavior disorder; (r) hallucinations; (s) fatigue; (t) apathy; (u) erectile dysfunction; (v) mood swings; (w) urinary incontinence; (x) mild cognitive impairment; and (y) neurodegeneration.

In some embodiments, the AD symptom is a sleep problem, sleep disorder, sleep disturbance, circadian rhythm dysfunction, REM disturbed sleep, or REM behavior disorder. In some embodiments: (a) the sleep disorder or sleep disturbance comprises a delay in sleep onset, sleep fragmentation, REM-behavior disorder, sleep-disordered breathing including snoring and apnea, day-time sleepiness, micro-sleep episodes, narcolepsy, hallucinations, or any combination thereof; (b) the REM-behavior disorder comprises vivid dreams, nightmares, and acting out the dreams by speaking or screaming, or fidgeting or thrashing of arms or legs during sleep; (c) the method results in a positive change in the sleeping pattern of the subject over a defined period of time; (d) the method results in a positive change in the sleeping pattern of the subject over a defined period of time, wherein the positive change is defined as: (i) an increase in the total amount of sleep obtained of at least about 5%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (e) as a result of the method the subject obtains the total number of hours of sleep recommended by a medical authority for the age group of the subject; and/or (f) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

In some embodiments, the AD symptom is a hallucination. In some embodiments: (a) the hallucination comprises a visual, auditory, tactile, gustatory or olfactory hallucination; (b) the method results in a decreased number of hallucinations over a defined period of time in the subject; (c) the method results in a decreased number of hallucinations over a defined period of time in the subject selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; (d) the method results in the subject being hallucination-free; (e) the method results in a decreased severity of hallucinations in the subject over a defined period of time, wherein the decrease in severity is measured by at least one medically-recognized technique; (f) the method results in a decreased severity of hallucinations in the subject over a defined period of time, wherein the decrease in severity is at least about 5% as measured by at least one medically recognized technique; (g) the at least one medically recognized technique is selected from the group consisting of Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA); and/or (h) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

In some embodiments, the AD symptom is depression. In some embodiments: (a) the method results in improvement in the subject's depression over a defined period of time, as measured by at least one clinically-recognized depression rating scale; (b) the method results in improvement in the subject's depression over a defined period of time, as measured by at least one clinically-recognized depression rating scale and the improvement is in at least one depression characteristic selected from the group consisting of mood, behavior, bodily functions such as eating, sleeping, energy, and sexual activity, and/or episodes of sadness or apathy; (c) the method results in improvement in the subject's depression over a defined period of time, as measured by at least one clinically-recognized depression rating scale, and the improvement a subject experiences following treatment is at least about 5%; and/or (d) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

In some embodiments, the AD symptom is cognitive impairment. In some embodiments: (a) progression or onset of the cognitive impairment is slowed, halted, or reversed over a defined period of time following administration of the composition, as measured by a medically-recognized technique; (b) the cognitive impairment is positively impacted by the administered composition, as measured by a medically-recognized technique; (c) the cognitive impairment is positively impacted by the administered composition, as measured by a medically-recognized technique and the positive impact on and/or progression of cognitive impairment is measured quantitatively or qualitatively by at least one technique selected from the group consisting of Mini-Mental State Exam (MMSE), Mini-cog test, Montreal Cognitive Assessment (MoCA), and a computerized test selected from Cantab Mobile, Cognigram, Cognivue, Cognision, and Automated Neuropsychological Assessment Metrics; (d) the progression or onset of cognitive impairment is slowed, halted, or reversed by at least about 5% as measured by a medically-recognized technique; and/or (e) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

In some embodiments, the Alzheimer's disease (AD) symptom is constipation. In some embodiments: (a) the composition causes the subject to have a bowel movement; (b) the method results in an increase in the frequency of bowel movement in the subject; and/or (c) the method results in an increase in the frequency of bowel movement in the subject and the increase in the frequency of bowel movement is defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (d) as a result of the method the subject has the frequency of bowel movement recommended by a medical authority for the age group of the subject.

In some embodiments, the AD symptom is neurodegeneration. In some embodiments: (a) the method results in treating, preventing, and/or delaying the progression and/or onset of neurodegeneration in the subject; (b) progression or onset of the neurodegeneration is slowed, halted, or reversed over a defined period of time following administration of the composition, as measured by a medically-recognized technique; (c) the neurodegeneration is positively impacted by the administered composition, as measured by a medically-recognized technique; (d) the progression of (b) and/or the positive impact of (c) is measured quantitatively or qualitatively by at least one technique selected from the group consisting of electroencephalogram (EEG), neuroimaging, functional MRI, structural MRI, diffusion tensor imaging (DTI), [18F]fluorodeoxyglucose (FDG) PET, agents that label amyloid beta or tau, [18F]F-dopa PET, radiotracer imaging, volumetric analysis of regional tissue loss, specific imaging markers of abnormal protein deposition, multimodal imaging, and biomarker analysis (plasma or cerebrospinal fluid); (e) the progression or onset of (b) is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or (f) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

Disclosed herein include compositions. In some embodiments, the composition comprises: a central nervous system (CNS) homing peptide comprising an amino acid sequence of any one of SEQ ID NOs: 1-22. In some embodiments, composition does not comprise any additional therapeutic agent.

Disclosed herein include compositions for use. In some embodiments, the composition comprises: a central nervous system (CNS) homing peptide comprising an amino acid sequence of any one of SEQ ID NOs: 1-22 for use in treating Alzheimer's disease (AD). In some embodiments, the composition does not comprise any additional therapeutic agent. In some embodiments, the composition comprises: a central nervous system (CNS) homing peptide comprising an amino acid sequence of any one of SEQ ID NOs: 1-22 for use in delaying or reducing the likelihood of onset of Alzheimer's disease (AD). In some embodiments, the composition does not comprise any additional therapeutic agent. In some embodiments, the composition comprises: a central nervous system (CNS) homing peptide comprising an amino acid sequence of any one of SEQ ID NOs: 1-22 for use in treating, preventing, or reversing cognitive decline in clinical or pre-clinical Alzheimer's disease (AD). In some embodiments, the composition does not comprise any additional therapeutic agent. In some embodiments, the composition comprises: a central nervous system (CNS) homing peptide comprising an amino acid sequence of any one of SEQ ID NOs: 1-22 for use in treating, delaying or reversing dementia. In some embodiments, the composition does not comprise any additional therapeutic agent. In some embodiments, the composition comprises: a central nervous system (CNS) homing peptide comprising an amino acid sequence of any one of SEQ ID NOs: 1-22 for use in delaying or reversing the progression of AD. In some embodiments, the composition does not comprise any additional therapeutic agent. In some embodiments, the composition is a pharmaceutical composition comprising the CNS homing peptide and at least one pharmaceutically acceptable excipient. In some embodiments, the composition is formulated for oral administration, intravenous administration, subcutaneous administration, or a combination thereof.

Disclosed herein include kits. In some embodiments, the kit comprises: at least one of a CNS homing peptide comprising an amino acid sequence of any one of SEQ ID NOs: 1-22; and a label indicating at least one of: (a) the kit is for preventing or delaying the onset of AD, (b) the kit is for treating AD, (c) the kit is for treating, preventing, or reversing cognitive decline in clinical or pre-clinical AD, (d) the kit is for delaying or reversing the progression of AD, and (e) the kit is for delaying, preventing, or reversing dementia. In some embodiments, the dementia is AD-related dementia. In some embodiments, kit does not comprise any additional therapeutic agent for any of: (a) preventing or delaying the onset of AD, (b) treating AD, (c) treating, preventing, or reversing cognitive decline in clinical or pre-clinical Alzheimer's disease (AD), (d) delaying or reversing the progression of AD; and (e) delaying, preventing, or reversing dementia. In some embodiments, the dementia is AD-related dementia. In some embodiments, the Alzheimer's disease (AD) is mild cognitive impairment associated with AD, early stage AD, mid-stage AD, and/or late-stage AD. In some embodiments, the AD is sporadic AD or familial AD.

### DETAILED DESCRIPTION

All patents, published patent applications, other publications, and sequences from GenBank, and other databases referred to herein are incorporated by reference in their entirety with respect to the related technology.

Disclosed herein include methods of treating, preventing, or reversing dementia in a subject in need thereof. In some embodiments, the method comprises administering to the subject a therapeutically effective amount of a composition comprising a central nervous system (CNS) homing peptide, thereby treating, preventing, or reversing dementia in the subject. In some embodiments, the composition does not comprise any additional therapeutic agent.

Disclosed herein include methods of treating Alzheimer's disease (AD), or delaying or reducing the likelihood of onset of AD. In some embodiments, the method comprises administering to a subject in need thereof a therapeutically effective amount of a composition comprising a central nervous system (CNS) homing peptide, thereby treating AD or delaying or reducing the likelihood of onset of AD in the subject. In some embodiments, the composition does not comprise any additional therapeutic agent.

Disclosed herein include methods of treating, preventing, or reversing cognitive decline in clinical or pre-clinical Alzheimer's disease (AD). In some embodiments, the method comprises administering to a subject in need thereof a therapeutically effective amount of a composition comprising a central nervous system (CNS) homing peptide, thereby treating, preventing, or reversing cognitive decline in clinical or pre-clinical AD in the subject. In some embodiments, the composition does not comprise any additional therapeutic agent.

Disclosed herein include methods of delaying or reversing the progression of Alzheimer's disease (AD). In some embodiments, the method comprises administering to a subject in need thereof a therapeutically effective amount of a composition comprising a central nervous system (CNS) homing peptide, thereby delaying or reversing the progression of AD in the subject. In some embodiments, the composition does not comprise any additional therapeutic agent.

Disclosed herein include compositions. In some embodiments, the composition comprises: a central nervous system (CNS) homing peptide comprising an amino acid sequence of any one of SEQ ID NOs: 1-22. In some embodiments, composition does not comprise any additional therapeutic agent.

Disclosed herein include compositions for use. In some embodiments, the composition comprises: a central nervous system (CNS) homing peptide comprising an amino acid sequence of any one of SEQ ID NOs: 1-22 for use in treating Alzheimer's disease (AD). In some embodiments, the composition comprises: a central nervous system (CNS) homing peptide comprising an amino acid sequence of any one of SEQ ID NOs: 1-22 for use in delaying or reducing the likelihood of onset of Alzheimer's disease (AD). In some embodiments, the composition comprises: a central nervous system (CNS) homing peptide comprising an amino acid sequence of any one of SEQ ID NOs: 1-22 for use in treating, preventing, or reversing cognitive decline in clinical or pre-clinical Alzheimer's disease (AD). In some embodiments, the composition comprises: a central nervous system (CNS) homing peptide comprising an amino acid sequence of any one of SEQ ID NOs: 1-22 for use in treating, delaying or reversing dementia. In some embodiments, the composition comprises: a central nervous system (CNS) homing peptide comprising an amino acid sequence of any one of SEQ ID NOs: 1-22 for use in delaying or reversing the progression of Alzheimer's disease (AD). In some embodiments, the compositions disclosed herein do not comprise any additional therapeutic agent.

Disclosed herein include kits. In some embodiments, the kit comprises: at least one of a central nervous system (CNS) homing peptide comprising an amino acid sequence of any one of SEQ ID NOs: 1-22; and a label indicating at least one of: (a) the kit is for preventing or delaying the onset of Alzheimer's disease (AD), (b) the kit is for treating AD, (c) the kit is for treating, preventing, or reversing cognitive decline in clinical or pre-clinical AD, (d) the kit is for delaying or reversing the progression of AD, and (e) the kit is for delaying, preventing, or reversing dementia (e.g., AD-related dementia).

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, N Y 1989). For purposes of the present disclosure, the following terms are defined below.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, the term “patient” refers to a subject having or suspected of having a disease. In some embodiments, the patient is a human or an animal. In some embodiments, the patient is a mammal.

As used herein, a “dosage” refers to the combined amount of the active ingredient (e.g., CNS homing peptide).

As used herein, a “unit dosage” refers to an amount of therapeutic agent administered to a patient in a single dose.

As used herein, a “daily dosage” refers to the total amount of therapeutic agent administered to a patient in a day.

As used herein, “therapeutic agent” or “pharmaceutically active ingredient” refers to a compound, which is suitable for or useful in preventing, modifying disease pathogenesis in a beneficial way, or treating a disease. As used herein, “therapeutically effective amount” or “pharmaceutically effective amount” refers to an amount of a therapeutic agent, which has a therapeutic effect. For example, in some embodiments, a therapeutically effective amount means an amount of therapeutic agent which produces the desired therapeutic effect as judged by clinical trial results and/or model animal studies. As used herein, the term “prophylactically effective amount” means an amount of a therapeutic agent which produces a prophylactic effect.

As used herein, a “therapeutic effect” relieves, to some extent, one or more of the symptoms of a disease or disorder. For example, in some embodiments, a therapeutic effect may be measured by one or more medically-recognized techniques.

As used herein, “treat,” “treatment,” “therapy,” or “treating” refers to administering a therapeutic agent or pharmaceutical composition to a subject for prophylactic and/or therapeutic purposes. “Treat”, “treatment”, or “treating”, as used herein, include alleviating or abrogating a disease or condition, or one or more symptoms associated with the disorder or condition, or alleviating or eradicating a cause(s) of the disorder or condition. As used herein, the term “prevent” or “preventing” refers to reducing or eliminating the onset of symptoms or complications of a disease or condition.

As used herein, “administration” or “administering” refers to a method of giving a dosage of a pharmaceutically active ingredient to a subject. Administration can be by any suitable route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery of pharmaceutical compositions and therapeutic substances disclosed herein include, but are not limited to, the use of liposomal formulations, intravenous infusion, transdermal patches, or a combination thereof.

As used herein, the term “delivery” refers to approaches, formulations, technologies, and systems for transporting a pharmaceutical composition or a therapeutic agent into the body of a patient as needed to safely achieve its desired therapeutic effect. In some embodiments, an effective amount of the composition or agent is formulated for delivery into the blood stream of a patient.

As used herein, the term “formulated” or “formulation” refers to the process in which different chemical substances, including one or more pharmaceutically active ingredients, are combined to produce a dosage form. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to a diseased tissue or a tissue adjacent to the diseased tissue. Carriers or excipients can be used to produce compositions. The carriers or excipients can be chosen to facilitate administration of a drug or pro-drug. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

### Central Nervous System (CNS) Homing Peptides

Provided herein are methods, compositions, and kits for treating, preventing, or reversing dementia and/or Alzheimer's disease (AD) in a subject in need thereof. In some embodiments, the method comprises administering to the subject a therapeutically effective amount of a composition comprising a central nervous system (CNS) homing peptide, thereby treating, preventing, or reversing dementia and/or AD in the subject. In some embodiments, the composition does not comprise any additional therapeutic agent.

Provided herein are compositions comprising a CNS homing or targeting peptide. The CNS homing or targeting peptide can comprise, or consist of, one of the amino acid sequences provided in Table 1. The CNS homing peptide can be capable of homing to the entorhinal cortex, the cerebral cortex, the hippocampus, or any combination thereof. In some embodiments, the CNS homing or targeting peptide comprises, or consists of, an amino acid sequence having one, two, three, four, or five amino acid mismatches as compared to one of the amino acid sequences provided in Table 1. In some embodiments, the CNS homing or targeting peptide comprises, or consists of, an amino acid sequence having a deletion of one, two, three, four, or five amino acids as compared to one of the amino acid sequences provided in Table 1. In some embodiments, the CNS homing or targeting peptide comprises, or consists of, an amino acid sequence having one, two, three, four, or five additional amino acids (e.g., additions to the N-terminal, internal additions, and additions to the C-terminal) as compared to one of the amino acid sequences provided in Table 1. CNS homing or targeting peptides have been described in U.S. Pat. No. 11,174,287, the content of which is incorporated herein by reference in its entirety.

TABLE 1
CNS HOMING OR TARGETING PEPTIDE SEQUENCES

| Amino Acid Sequence | SED ID NO |
|---|---|
| KRSS | 1 |
| PGESS | 2 |
| SLTQ | 3 |
| AMGN | 4 |
| GDRLV | 5 |
| RGGKRSS | 6 |
| SLTQDQG | 7 |
| ADAQADV | 8 |
| RAKGRDA | 9 |
| KASRLGR | 10 |
| RGASMDG | 11 |
| GRPGESS | 12 |
| SRTEGDV | 13 |
| CRGGKRSSC | 14 |
| CSLTQDQGC | 15 |
| CADAQADVC | 16 |
| CRAKGRDAC | 17 |
| CKASRLGRC | 18 |
| CRGASMDGC | 19 |
| CGRPGESSC | 20 |
| CSRTEGDVC | 21 |
| CSGRRGKSC | 22 |

Methods to Identify Subjects at Risk of or Having Dementia and/or Alzheimer's Disease

Dementia (e.g., major neurocognitive disorder) is defined by chronic, acquired loss of one or two or more cognitive abilities caused by brain disease or injury. Cognitive domains can include memory, language, visuospatial, executive, or other. Loss of cognitive ability typically represents a decline from the prior level of function and impairs function abilities in day-to-day life. The most common cause of dementia is Alzheimer's disease (AD), accounting for 60 to 80 percent of cases. Memory requires the recording, storage, and retrieval of information. The most common clinical presentation of AD is a slow onset and gradually progressive loss of memory, typically with inability to learn new information and particularly autobiographical information, such as recent events in ones' life. This is because AD preferentially affects brain networks involved in episodic memory. Examples of episodic memory loss include forgetting appointments, to pay bills or to take medication. Typically, a person with AD repeats questions and conversations. The memory loss is often accompanied by subjective memory complaints. Difficulty recalling names which are recalled later, is common in aging but is not a typical early sign of dementia. Mild cognitive impairment (MCI) is defined by performance that is lower than normal on objective neuropsychological testing of cognition, but with maintained daily functions (e.g., maintained abilities to function within society such as for daily activities at work, home, and in social settings, and maintained activities of daily living such as for personal care) and therefore not consistent with dementia. MCI can be categorized into “amnestic” MCI, in which reduced performance on memory is the key finding, versus “non-amnestic” MCI, in which reduced cognitive performance is in a non-memory domain such as language. MCI can also be characterized into “single domain” versus “multi-domain” MCI, in which multiple cognitive performance measures are impaired. MCI does not always progress to dementia, and a patient's cognitive status may become normal or fluctuate between MCI, normal cognition, and dementia. Fluctuations in cognition are also present in some conditions including neurodegenerative diseases (such as in early stages of Lewy body disease), cerebrovascular disease (e.g., intermittent small strokes), and psychiatric conditions (e.g., depression, anxiety), and with medications affecting cognition (e.g., opioids), and variability in cognitive test results.

In some embodiments, the subject is suspected of having dementia, is diagnosed with dementia, or has dementia. The dementia can be Alzheimer's disease (AD)-related dementia, vascular dementia, Lewy body dementia, fronto-temporal dementia, or mixed dementia. Other diseases which can cause dementia include normal pressure hydrocephalus, Creutzfeldt-Jakob disease, Huntington's Disease, chronic traumatic encephalopathy (CTE), HIV, heavy alcohol use over time, head injury (e.g., concussion), emotional problems (e.g., stress, anxiety, and depression), and delirium (e.g., sudden state of confusion and disorientation).

AD-related dementia is caused by specific changes in the brain. The trademark symptom is trouble remembering recent events, such as a conversation that occurred minutes or hours ago, while difficulty remembering more distant memories occurs later in the disease. Other concerns like difficulty with walking or talking or personality changes also come later. Family history is the most important risk factor. Having a first-degree relative with Alzheimer's disease increases the risk of developing it by 10 to 30 percent. In some embodiments, the dementia is vascular dementia. About 10 percent of dementia cases are linked to strokes or other issues with blood flow to the brain. Diabetes, high blood pressure and high cholesterol are also risk factors. Symptoms vary depending on the area and size of the brain impacted. The disease progresses in a step-wise fashion, meaning symptoms will suddenly get worse as the individual gets more strokes or mini-strokes. In some embodiments, the dementia is Lewy body dementia. In addition to more typical symptoms like memory loss, people with this form of dementia may have movement or balance problems like stiffness or trembling. Many people also experience changes in alertness including daytime sleepiness, confusion or staring spells. They may also have trouble sleeping at night or may experience visual hallucinations (seeing people, objects or shapes that are not actually there). In some embodiments, the dementia is fronto-temporal dementia. This type of dementia most often leads to changes in personality and behavior because of the part of the brain it affects. People with this condition may embarrass themselves or behave inappropriately. For instance, a previously cautious person may make offensive comments and neglect responsibilities at home or work. There may also be problems with language skills like speaking or understanding. In some embodiments, the dementia is mixed dementia. Sometimes more than one type of dementia is present in the brain at the same time, especially in people aged 80 and older. For example, a person may have Alzheimer's disease and vascular dementia. It is not always obvious that a person has mixed dementia since the symptoms of one type of dementia may be most prominent or may overlap with symptoms of another type. Disease progression may be faster than with one kind of dementia.

In some embodiments, more than one method is used to diagnose dementia. Methods for diagnosing dementia are described in Arvanitakis et al., 2019 “Diagnosis and management of dementia” JAMA, 322(16): 1589-1599, which is hereby incorporated by reference in its entirety. Dementia can be identified based on one or more of: medical history, including from family, friend, or caregiver, focusing on cognition and function; brief outpatient or bedside cognitive examination; and neuropsychological testing. The cause of dementia can be determined by, for example, examination of medical history (e.g., neurologic history, general medical history, and/or family history), a physical examination (e.g., for neurological signs such as cognitive impairment, focal signs, parkinsonism, etc.; and systemic signs such as vascular and metabolic diseases), and laboratory testing for, e.g., thyroid function and vitamin B12. Additional tests can include, but are not limited to: computed tomography (CT) or magnetic resonance imaging (MRI) scan, positron emission tomography (PET), electroencephalogram (EEG), magnetic resonance angiogram (MRA), computed tomography angiogram (CTA), and electrocardiogram (ECG).

The cognitive examination can identify the presence, severity and nature of impairment. Multiple medically recognized techniques are available for assessment of impairment. For example, one method is the Montreal Cognitive Assessment (MoCA; range 0-30, follow-up evaluation to screening recommended if score <24/30). The MoCA requires about 10 minutes to administer and is useful in early detection of cognitive impairment, including MCI with executive dysfunction. The Mini-Mental State Exam was developed more than 4 decades ago. It is less sensitive to the presence of MCI and less thoroughly evaluates the domains of executive function, higher-level language skills, and complex visuospatial processing. The subject can also be assessed for other neurological symptoms such as aphasia, ataxia, and agnosia. Methods for assessing cognitive impairment can also include Mini-cog test, or a computerized test selected from Cantab Mobile, Cognigram, Cognivue, Cognision, or Automated Neuropsychological Assessment Metrics.

The Diagnostic and Statistical Manual of Mental Disorders, Fifth edition (DSM-V) diagnostic criteria for dementia (e.g., major neurocognitive disorder) include: a) Evidence of significant cognitive decline from a previous level of performance in one or more cognitive domains (e.g., executive function, learning and memory, social cognition, and others); b) The cognitive defects interfere with independence in everyday activities; c) The cognitive defects do not occur exclusively in the context of delirium; d) The cognitive deficits are not better explained by another mental disorder (e.g., behavioral disturbance). The cognitive impairment of a) can be determined by any of the methods disclosed herein, e.g., MoCA or MMSE test or any neuropsychological test known to a skilled artisan. In some embodiments, a subject having dementia will perform at least about 2 standard deviations below the normative mean on the neuropsychological test. As noted above, a brief bedside assessment by a clinician or other individual skilled in the art can also be performed.

As discussed above, AD is the most common cause of dementia. In some embodiments, the subject in need is suspected of having AD. The method can comprise identifying the subject in need thereof, wherein the subject in need thereof is a subject at a risk of having Alzheimer's disease (AD), a subject having AD, or a subject suspected of having AD. The method can comprise identifying the subject in need thereof as a subject having mild cognitive impairment or dementia associated with Alzheimer's disease (AD), early stage AD, mid-stage AD, late-stage AD, sporadic AD, or familial AD. A subject in need of identifying the presence of AD phenotype is any subject at risk of, or suspected of, having AD. A subject at risk of having AD can be, for example, a subject having one or more risk factors for AD. Risk factors for AD include, but are not limited to, age, family history, heredity and brain injury. Other risk factors will be apparent to the skilled artisan. A subject suspected of having AD can be, for example, a subject having one or more clinical symptoms of AD. A variety of clinical symptoms of AD are known in the art. Examples of such symptoms include, but are not limited to, memory loss, depression, anxiety, language disorders (e.g., anomia) and impairment in their visuospatial skills. Some embodiments of the methods provided herein comprise one or more patient identification steps (e.g., identifying subjects amenable to treatment using the methods as disclosed herein). Some embodiments of the methods provided herein comprise identifying subsets of subjects suitable for treatment using the methods as disclosed herein.

Subjects amenable to treatment using the methods as disclosed herein include subjects at risk of a neurodegenerative disease, for example Alzheimer's disease but not showing symptoms, as well as subjects showing symptoms of the neurodegenerative disease, for example subjects with symptoms of Alzheimer's disease. Subjects can be screened for their likelihood of having or developing Alzheimer's disease based on a number of biochemical and genetic markers.

In some embodiments, one can diagnose a subject with increased risk of developing Alzheimer's disease using genetic markers for Alzheimer's disease. The subject in need thereof can have at least one mutation in one or more genes associated with Alzheimer's disease. The one or more genes can comprise apolipoprotein E (APOE), amyloid precursor protein (APP), presenilin 1 (PSEN1), and/or presenilin 2 (PSEN2). Genetic abnormality in a few families has been traced to chromosome 21 (St. George-Hyslop et al., Science 235:885-890, 1987). One genetic marker is, for example, mutations in the APP gene, particularly mutations at position 717 and positions 670 and 671 referred to as the Hardy and Swedish mutations respectively (see Hardy, TINS, supra). Other markers of risk are mutations in the presenilin genes, PSEN1 and PSEN2, and ApoE4, family history of Alzheimer's disease, hypercholesterolemia or atherosclerosis. Subjects with APP, PSEN1 or PSEN2 mutations are highly likely to develop Alzheimer's disease. ApoE is a susceptibility gene, and subjects with the e4 isoform of ApoE (ApoE4 isoform) have an increased risk of developing Alzheimer's disease. Test for subjects with ApoE4 isoform are disclosed in U.S. Pat. No. 6,027,896, which is incorporated in its entirety herein by reference. Other genetic links have been associated with increased risk of Alzheimer's disease, for example variances in the neuronal sortilin-related receptor SORL1 may have increased likelihood of developing late-onset Alzheimer's disease (Rogaeva at al., Nat Genet. 2007 February; 39(2):168-77). Other potential Alzheimer's disease susceptibility genes, include, for example ACE, CHRNB2, CST3, ESR1, GAPDHS, IDE, MTHFR, NCSTN, PRNP, TF, TFAM and TNF and be used to identify subjects with increased risk of developing AD (Bertram et al, Nat Genet. 2007 January; 39(1): 17-23), as well as variances in the alpha-T catenin (VR22) gene (Bertram et al, J Med Genet. 2007 January; 44(1):e63) and Insulin-degrading enzyme (IDE) and Kim et al, J Biol Chem. 2007; 282:7825-32).

In some embodiments, one can diagnose a subject with increased risk of developing Alzheimer's disease on the basis of a simple eye test, where the presence of cataracts and/or amyloid beta in the lens identifies a subject with increased risk of developing Alzheimer's disease. Methods to detect Alzheimer's disease include using a quasi-elastic light scattering device (Goldstein et al., Lancet. 2003; 12; 361:1258-65) from Neuroptix, using Quasi-Elastic Light Scattering (QLS) and Fluorescent Ligand Scanning (FLS) and a Neuroptix™ QEL scanning device, to enable non-invasive quantitative measurements of amyloid aggregates in the eye, to examine and measure deposits in specific areas of the lens as an early diagnostic for Alzheimer's disease. Methods to diagnose a subject at risk of developing Alzheimer's disease using such a method of non-invasive eye test are disclosed in U.S. Pat. No. 7,107,092, which is incorporated in its entirety herein by reference.

Individuals presently suffering from Alzheimer's disease can be recognized, for example, from characteristic dementia, as well as the presence of risk factors described above. In addition, a number of diagnostic tests are available for identifying individuals who have AD. These include measurement of cerebrospinal fluid (CSF) tau and Ax3b242 levels. Elevated tau and decreased Ax3b242 levels signify the presence of Alzheimer's Disease.

There are at least two alternative “criteria” which are utilized to clinically diagnose Alzheimer's disease: the DSM-IIIR criteria and the NINCDS-ADRDA criteria (which is an acronym for National Institute of Neurological and Communicative Disorders and Stroke (NINCDS) and the Alzheimer's Disease and Related Disorders Association (ADRDA); see McKhann et al., Neurology 34:939-944, 1984). Briefly, the criteria for diagnosis of Alzheimer's Disease under DSM-IIIR include (1) dementia, (2) insidious onset with a generally progressive deteriorating course, and (3) exclusion of all other specific causes of dementia by history, physical examination, and laboratory tests. Within the context of the DSM-IIIR criteria, dementia is understood to involve “a multifaceted loss of intellectual abilities, such as memory, judgment, abstract thought, and other higher cortical functions, and changes in personality and behaviour.” (DSM-IIR, 1987).

The NINCDS-ADRDA criteria set forth three categories of Alzheimer's Disease, including “probable,” “possible,” and “definite” AD. Clinical diagnosis of “possible” Alzheimer's disease may be made on the basis of a dementia syndrome, in the absence of other neurologic, psychiatric or systemic disorders sufficient to cause dementia. Criteria for the clinical diagnosis of “probable” AD include (a) dementia established by clinical examination and documented by a test such as the Mini-Mental test (Foldstein et al., J. Psych. Res. 12:189-198, 1975); (b) deficits in two or more areas of cognition; (c) progressive worsening of memory and other cognitive functions; (d) no disturbance of consciousness; (e) onset between ages 40 and 90, most often after age 65; and (f) absence of systemic orders or other brain diseases that could account for the dementia. The criteria for definite diagnosis of AD include histopathologic evidence obtained from a biopsy, or after autopsy. Since confirmation of definite AD requires histological examination from a brain biopsy specimen (which is often difficult to obtain), it is rarely used for early diagnosis of Alzheimer's disease.

In some embodiments, one can use neuropathologic diagnosis of AD, where the numbers of plaques and tangles in the neurocortex (frontal, temporal, and parietal lobes), hippocampus and amygdala are analyzed (Khachaturian, Arch. Neurol. 42:1097-1105; Esiri, “Anatomical Criteria for the Biopsy diagnosis of Alzheimer's Disease,” Alzheimer's Disease, Current Research in Early Diagnosis, Becker and Giacobini (eds.), pp. 239-252, 1990).

In some embodiments, one can use quantitative electroencephalographic analysis (EEG) to diagnose AD. This method employs Fourier analysis of the beta, alpha, theta, and delta bands (Riekkinen et al., “EEG in the Diagnosis of Early Alzheimer's Disease,” Alzheimer's Disease, Current Research in Early Diagnosis, Becker and Giacobini (eds.), pp. 159-167, 1990) for diagnosis of Alzheimer's Disease.

In some embodiments, one can diagnose AD by quantifying the degree of neural atrophy, since such atrophy is generally accepted as a consequence of AD. Examples of these methods include computed tomographic scanning (CT), and magnetic resonance imaging (MRI) (Leedom and Miller, “CT, MRI, and NMR Spectroscopy in Alzheimer's Disease,” Alzheimer's Disease, Current Research in Early Diagnosis, Becker and Giacobini (eds.), pp. 297-313, 1990).

In some embodiments, one can diagnose AD by assessing decreased cerebral blood flow or metabolism in the posterior temporoparietal cerebral cortex by measuring decreased blood flow or metabolism by positron emission tomography (PET) (Parks and Becker, “Positron Emission Tomography and Neuropsychological Studies in Dementia,” Alzheimer's Disease's, Current Research in Early Diagnosis, Becker and Giacobini (eds.), pp. 315-327, 1990), single photon emission computed tomography (SPECT) (Mena et al., “SPECT Studies in Alzheimer's Type Dementia Patients,” Alzheimer's Disease, Current Research in Early Diagnosis, Becker and Giacobini (eds.), pp. 339-355, 1990), and xenon inhalation methods (Jagust et al., Neurology 38:909-912; Prohovnik et al., Neurology 38:931-937; and Waldemar et al., Senile Dementias: II International Symposium, pp. 399407, 1988).

In some embodiments, one can immunologically diagnose AD (Wolozin, “Immunochemical Approaches to the Diagnosis of Alzheimer's Disease,” Alzheimer's Disease, Current Research in Early Diagnosis, Becker and Giacobini (eds.), pp. 217-235, 1990). Wolozin and coworkers (Wolozin et al., Science 232:648-650, 1986) produced a monoclonal antibody “Alz50,” that reacts with a 68-kDa protein “A68,” which is expressed in the plaques and neuron tangles of patients with AD. Using the antibody Alz50 and Western blot analysis, A68 was detected in the cerebrospinal fluid (CSF) of some AD patients and not in the CSF of normal elderly patients (Wolozin and Davies, Ann. Neurol. 22:521-526, 1987).

In some embodiments, one can diagnose AD using neurochemical markers of AD. Neurochemical markers which have been associated with Alzheimer's Disease include reduced levels of acetylcholinesterase (Giacobini and Sugaya, “Markers of Cholinergic Dysfunction in Alzheimer's Disease,” Alzheimer's Disease, Current Research in Early Diagnosis, Becker and Giacobini (eds.), pp. 137-156, 1990), reduced somatostatin (Tamminga et al., Neurology 37:161-165, 1987), a negative relation between serotonin and 5-hydroxyindoleacetic acid (Volicer et al., Arch Neurol. 42:127-129, 1985), greater probenecid-induced rise in homovanyllic acid (Gibson et al., Arch. Neurol. 42:489-492, 1985) and reduced neuron-specific enolase (Cutler et al., Arch. Neurol. 43:153-154, 1986).

In some embodiments, the subject is diagnosed with mild cognitive impairment associated with Alzheimer's disease (AD), early stage AD, mid-stage AD, or late-stage AD. In some embodiments, the subject is diagnosed with AD-related dementia. The Alzheimer's disease can be sporadic Alzheimer's disease or familial AD.

### Methods of Treating and Preventing Dementia and Alzheimer's Disease

Disclosed herein include methods of treating, preventing, or reversing dementia in a subject in need thereof. Provided herein are methods of treating AD, or delaying or reducing the likelihood of onset of AD. The method can comprise administering to a subject in need thereof a therapeutically effective amount of a composition comprising a CNS homing peptide. In some embodiments, the composition comprises a therapeutically or prophylactically effective amount of a CNS homing peptide. In some embodiments, the composition does not comprise any additional therapeutic agent. In some embodiments, the subject is a mammal. In some embodiments, the subject is a human. In some embodiments, said subject is a human over 40 years old, over 50 years old, over 60 years old, over 70 years old, over 80 years old, and/or over 85 years old.

In some embodiments, the subject in need has dementia, is suspected of having dementia, or is at risk of having dementia. The dementia can be AD-related dementia, vascular dementia, Lewy body dementia, fronto-temporal dementia, or mixed dementia. In some embodiments, the subject in need thereof has AD, is suspected of having AD, or is at a risk of having AD. In some embodiments, said subject is diagnosed with mild cognitive impairment potentially related to AD, early stage AD, mid-stage AD, or late-stage AD. In some embodiments, said AD is sporadic (non-hereditary) AD. In some embodiments, said AD is familial (hereditary) AD. Diagnosis of different types of AD and one or more symptoms associated with AD can be conducted by methods and techniques known in the art. For example, diagnosis of mild cognitive impairment due to Alzheimer's disease can be performed as described in Alert et al. Alzheimers Dement. 2011 May; 7(3): 270-279. doi:10.1016/j.jalz.2011.03.008, the content of which is incorporated herein by reference in its entirety. The diagnosis of mild cognitive impairment due to Alzheimer's disease, in some embodiments, includes two sets of criteria: (1) core clinical criteria that could be used by healthcare providers without access to advanced imaging techniques or cerebrospinal fluid analysis, and (2) research criteria that could be used in clinical research settings, including clinical trials. The second set of criteria incorporate the use of biomarkers based on imaging and cerebrospinal fluid measures. The final set of criteria for mild cognitive impairment due to AD has four levels of certainty, depending on the presence and nature of the biomarker findings. In some embodiments, the methods and compositions disclosed herein are used to treat and relieve one or more symptoms related to AD, including mild cognitive impairment potentially related to AD. In some embodiments, the subject suffers from Mild Cognitive Impairment (MCI), for example subject suffering from MCI and has an abnormal brain positron emission tomography (PET) scan or spinal fluid test for amyloid beta protein, which is the protein in amyloid plaques (one of the two hallmarks of Alzheimer's). In some embodiments, the subject is a subject diagnosed of MCI due to Alzheimer's disease. In some embodiments, the methods and compositions disclosed herein are used to treat MCI.

Disclosed herein include methods of treating, preventing, or reversing dementia in a subject in need thereof. In some embodiments, the method comprises administering to the subject a therapeutically effective amount of a composition comprising a central nervous system (CNS) homing peptide, thereby treating, preventing, or reversing dementia in the subject. In some embodiments, the composition does not comprise any additional therapeutic agent.

The dementia can be AD-related dementia, vascular dementia, Lewy body dementia, fronto-temporal dementia, or mixed dementia. In some embodiments, the dementia is prevented from occurring. In some embodiments, the onset of dementia is delayed. The delay can be, for example, days, weeks, months, or years. In some embodiments, the onset of dementia is delayed by at least, or at least about, one, two, three, four, five, six, seven, eight, nine, ten, or more weeks. In some embodiments, the onset of dementia is delayed by at least, or at least about, one, two, three, four, five, six, seven, eight, nine, ten, eleven, twelve, or more months. In some embodiments, the onset of dementia is delayed by at least, or at least about, one, two, three, four, five, six, seven, eight, nine, ten, eleven, twelve, or more years. One or more methods described herein for detecting/diagnosing dementia and one or more symptoms associated with dementia can be used to determine the delay in the onset of dementia.

There are provided, in some embodiments, methods of treating AD, or delaying or reducing the likelihood of onset of AD. In some embodiments, the method comprises: administering to a subject in need thereof a composition comprising CNS homing peptide, thereby delaying or reducing the likelihood of onset of AD in the subject, wherein the subject in need thereof is a subject that is at a risk of suffering from AD. In some embodiments, AD is prevented from occurring. In some embodiments, the onset of AD is delayed. The delay can be, for example, days, weeks, months, or years. In some embodiments, the onset of AD is delayed by at least, or at least about, one, two, three, four, five, six, seven, eight, nine, ten, or more weeks. In some embodiments, the onset of AD is delayed by at least, or at least about, one, two, three, four, five, six, seven, eight, nine, ten, eleven, twelve, or more months. In some embodiments, the onset of AD is delayed by at least, or at least about, one, two, three, four, five, six, seven, eight, nine, ten, eleven, twelve, or more years. One or more methods described herein for detecting/diagnosing AD and one or more symptoms associated with AD can be used to determine the delay in the onset of AD.

Disclosed herein include methods of treating, preventing, or reversing cognitive decline in clinical or pre-clinical AD. In some embodiments, the method comprises administering to a subject in need thereof a therapeutically effective amount of a composition comprising a CNS homing peptide, thereby treating, preventing, or reversing cognitive decline in clinical or pre-clinical AD in the subject. In some embodiments, the composition does not comprise any additional therapeutic agent.

Disclosed herein include methods of delaying or reversing the progression of AD. In some embodiments, the method comprises administering to a subject in need thereof a therapeutically effective amount of a composition comprising a CNS homing peptide, thereby delaying or reversing the progression of AD in the subject. In some embodiments, the composition does not comprise any additional therapeutic agent.

In some embodiments, the subject is not receiving any additional treatments or therapies. In some embodiments, the subject is not being administered any additional therapeutic agent (e.g., no additional therapeutic agent is administered concurrently with the composition comprising the CNS homing peptide). In some embodiments, the subject is not receiving any additional therapy for treating, preventing, or reversing dementia. In some embodiments, the subject is not being administered any additional therapeutic agent for treating, preventing, or reversing dementia. In some embodiments, the subject is not receiving any additional therapy for treating AD or delaying or reducing the likelihood of onset of AD. In some embodiments, the subject is not being administered any additional therapeutic agent for treating AD or delaying or reducing the likelihood of onset of AD. In some embodiments, the subject is not receiving any additional therapy for treating, preventing, or reversing cognitive decline in clinical or pre-clinical AD. In some embodiments, the subject is not being administered any additional therapeutic agent for treating, preventing, or reversing cognitive decline in clinical or pre-clinical AD. In some embodiments, the subject is not receiving any additional therapy for delaying or reversing the progression of AD. In some embodiments, the subject is not being administered any additional therapeutic agent for delaying or reversing the progression of AD. In some embodiments, additional therapies are discontinued prior to administration of the composition comprising the CNS homing peptide.

In some embodiments, the additional therapy, treatment, or therapeutic agent comprises an antibody capable of binding to amyloid beta or fragments thereof, an acetylcholinesterase inhibitor, an N-Methyl-D-Aspartic acid (NMDA) receptor antagonist or any combination thereof. In some embodiments, the additional therapy, treatment, or therapeutic agent comprises an amyloid lowering or inhibiting agent, an alpha-adrenergic receptor agonist, a beta-adrenergic receptor blocking agent (beta blocker), an anticholinergic, an anticonvulsant, an antipsychotic, a calcium channel blocker, a catechol O-methyltransferase (COMT) inhibitor, a central nervous system stimulant, a corticosteroid, a dopamine receptor agonist, a dopamine receptor antagonist, a dopamine reuptake inhibitor, a gamma-amino-butyric acid (GABA) receptor agonist, a histamine 3 (H3) antagonist, an immunomodulator, an immunosuppressant, an interferon, a monoamine oxidase (MAO) inhibitor, a muscarinic receptor agonist, a neuroprotective drug, a nicotinic receptor agonist, a norepinephrine (noradrenaline) reuptake inhibitor, a phosphodiesterase (PDE) inhibitor, a B-secretase inhibitor, a serotonin (5-hydroxytryptamine) 1A (5-HT1A) receptor antagonist, a serotonin (5-hydroxytryptamine) 2C (5-HT2c) receptor agonist, a serotonin (5-hydroxytryptamine) 4 (5-HT4) receptor agonist, a serotonin (5-hydroxytryptamine) 6 (5-HT6) receptor antagonist, a serotonin (5-HT) reuptake inhibitor, a trophic factor, a Glycine transporter-1 inhibitor, an AMPA-type glutamate receptor, a Janus kinase inhibitor (JAK), and an Interleukin-1 receptor-associated kinase 4 inhibitor (IRAK4). In some embodiments, the additional therapeutic agent comprises an non-steroidal anti-inflammatory drug (NSAID).

In some embodiments, administration of a CNS homing peptide as disclosed herein reduces or prevents neurogenic inflammation in the brain. “Neurogenic inflammation,” as used herein, shall be given its ordinary meaning, and includes the local release of inflammatory mediators from afferent neurons such as substance P and calcitonin gene-related peptide and/or their associated downstream effects. The terms “inflammation” and “inflammatory response” shall be given their ordinary meaning, and also include immune-related responses and/or allergic reactions to a physical, chemical, or biological stimulus. Measuring brain inflammation can comprise measuring the level of a pro-inflammatory cytokine, an anti-inflammatory cytokine, or a combination of pro-inflammatory cytokines and anti-inflammatory cytokines. Brain inflammation can comprise mast cell degranulation and plasma extravasation. Administering the composition can result in an at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or a range or a number between any two of these values) reduction of one or more of mast cell degranulation and plasma extravasation. In some embodiments of the methods and compositions provided herein, lymphopenia and/or mononuclear cell infiltration in the brain is reduced by at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or a range or a number between any two of these values).

The method can comprise measuring one or more dementia symptom in the subject before administering the composition to the subject, after administering the composition to the subject, or both. In some embodiments, administering the composition treats, prevents, improves, and/or resolves one or more dementia symptom. In some embodiments, administering the composition treats, prevents, improves, and/or resolves one or more dementia symptom by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or a range or a number between any two of these values. In some embodiments, the progression or onset of dementia is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a range or a number between any two of these values. In some embodiments, the progression or onset of dementia is evaluated by measuring one or more symptoms of dementia. In some embodiments, the progression or onset of dementia is measured quantitatively or qualitatively by one or more techniques selected from the group comprising Montreal Cognitive Assessment (MoCA), Mini-Mental State Exam (MMSE), Mini-cog test, and a computerized test selected from Cantab Mobile, Cognigram, Cognivue, Cognision, or Automated Neuropsychological Assessment Metrics. In some embodiments, the subject is assessed for cognitive criteria before, during and after administration of the composition.

Administering the composition comprising a CNS homing peptide can result in an at least, or at least about, 2% (e.g., at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 40%, at least about 50%, at least about 75%, at least about 100%, or a range or a number between any two of these values) reduction of one or more of (i) formation of plaques, (ii) amyloid fibril formation, (iii) amyloid-induced cellular toxicity or microglial activation, (iv) amyloid-induced neurotoxicity, (v) the rate or amount of amyloid aggregation, (vi) fibril formation, or deposition, (vii) the degree of amyloid deposition, (viii) amyloid-induced inflammation, (ix) neuroinflammation, (x) neurofibrillary tangles (NFTs, which are tangles containing hyperphosphorylated tau), and (xi) concentration of phosphorylated tau in the brain of the subject. Disclosed herein include methods for clinically assessing the effect of a composition comprising a CNS homing peptide on tau neurofibrillary tangles and/or phosphorylated tau. In some embodiments, the methods disclosed herein include a step of assessing the subject in need of treatment on tau neurofibrillary tangles and/or phosphorylated tau, and the step can be prior to, during, and after the treatment to the subject. In some embodiments, the methods and composition disclosed herein can reduce or slow down the formation of neurofibrillary tangles (i.e., tangles containing hyperphosphorylated tau). In some embodiments, the methods and composition disclosed herein can reduce the amount of neurofibrillary tangles or to reverse neurofibrillary tangle formation. In some embodiments, the methods and compositions disclosed herein can reduce the concentration or the amount of phosphorylated tau.

In some embodiments, the reduction of neuroinflammation comprises changes in one or more of cell signaling molecule production, activation of glia or glial activation pathways and responses, proinflammatory cytokines or chemokines, oxidative stress-related responses, acute phase proteins, components of the complement cascade, protein kinase activity, cell damage and cell death signal transduction pathways.

The method can comprise measuring one or more AD symptom in the subject before administering the composition to the subject, after administering the composition to the subject, or both. In some embodiments, administering the composition treats, prevents, improves, and/or resolves one or more AD symptom. In some embodiments, administering the composition treats, prevents, improves, and/or resolves one or more AD symptom by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or a range or a number between any two of these values. In some embodiments, the progression or onset of AD is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a range or a number between any two of these values. In some embodiments, the progression or onset of AD is evaluated by measuring one or more symptoms of AD. In some embodiments, the progression or onset of AD is measured quantitatively or qualitatively by one or more techniques selected from the group comprising electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, biomarker analysis, or any combination thereof.

The AD symptom can be, for example, (a) a symptom from the Integrated Alzheimer's Disease Rating Scale (iADRS) selected from personal belonging management, selection of clothes, ability to dress self, ability to clean habitation, financial management ability, writing ability, ability to keep appointments, ability to use telephone, ability to prepare food for self, travel ability, awareness of current events, reading ability, interest in television, ability to shop for self, ability to remain alone, ability to perform chores, ability to perform a hobby or game, driving ability, self-management of medications, ability to initiate and finish complex tasks, and ability to initiate and finish simple tasks; (b) a symptom from the Alzheimer's Disease Assessment Scale-Cognitive subscale (ADAS-Cog) selected from learning, naming, command following, ideational praxis, constructional praxis, orientation, and recognition memory; (c) a symptom from the Alzheimer's Disease Cooperative Study-instrumental Activities of Daily Living (ADCS-iADL) wherein the symptom is any of the symptoms recited in (a) or (b); (d) constipation; (e) depression; (f) cognitive impairment; (g) short term memory impairment; (h) long term memory impairment; (i) concentration impairment; (j) coordination impairment; (k) mobility impairment; (l) speech impairment; (m) mental confusion; (n) sleep problem, sleep disorder, or sleep disturbance; (o) circadian rhythm dysfunction; (p) REM disturbed sleep; (q) REM behavior disorder; (r) hallucinations; (s) fatigue; (t) apathy; (u) erectile dysfunction; (v) mood swings; (w) urinary incontinence; (x) mild cognitive impairment; (y) neurodegeneration; or any combination thereof. In some embodiments, administering the composition comprising a CNS homing peptide results in an at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or a range or a number between any two of these values) reduction of one or more AD symptom of the subject as compared to that in the subject before administration of the composition.

The one or more AD symptom can comprise a sleep problem, sleep disorder, sleep disturbance, circadian rhythm dysfunction, REM disturbed sleep, or REM behavior disorder. In some embodiments, (a) the sleep disorder or sleep disturbance comprises a delay in sleep onset, sleep fragmentation, REM-behavior disorder, sleep-disordered breathing including snoring and apnea, day-time sleepiness, micro-sleep episodes, narcolepsy, hallucinations, or any combination thereof; (b) the REM-behavior disorder comprises vivid dreams, nightmares, and acting out the dreams by speaking or screaming, or fidgeting or thrashing of arms or legs during sleep; (c) the method results in a positive change in the sleeping pattern of the subject over a defined period of time; (d) the method results in a positive change in the sleeping pattern of the subject over a defined period of time, wherein the positive change is defined as: (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a range or a number between any two of these values; (e) as a result of the method the subject obtains the total number of hours of sleep recommended by a medical authority for the age group of the subject; and/or (e) each defined period of time is independently selected from about 1 day to about 10 days, about 10 days to about 30 days, about 30 days to about 3 months, about 3 months to about 6 months, about 6 months to about 12 months, and about greater than 12 months.

The one or more AD symptom can comprise a hallucination(s). In some embodiments, (a) the hallucination comprises a visual, auditory, tactile, gustatory or olfactory hallucination; (b) the method results in a decreased number of hallucinations over a defined period of time in the subject; (c) the method results in a decreased number of hallucinations over a defined period of time in the subject selected from by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; (d) the method results in the subject being hallucination-free; (e) the method results in a decreased severity of hallucinations in the subject over a defined period of time, wherein the decrease in severity is measured by one or more medically-recognized techniques; (f) the method results in a decreased severity of hallucinations in the subject over a defined period of time, wherein the decrease in severity is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, or a range or a number between any two of these values, as measured by one or more medically recognized techniques; (g) the one or more medically recognized techniques is selected from Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA); and/or (h) each defined period of time is independently selected from about 1 day to about 10 days, about 10 days to about 30 days, about 30 days to about 3 months, about 3 months to about 6 months, about 6 months to about 12 months, and about greater than 12 months.

The one or more AD symptom can comprise depression. In some embodiments, (a) the method results in improvement in the subject's depression over a defined period of time, as measured by one or more clinically-recognized depression rating scale; (b) the method results in improvement in the subject's depression over a defined period of time, as measured by one or more clinically-recognized depression rating scale and the improvement is in one or more depression characteristics selected from mood, behavior, bodily functions such as eating, sleeping, energy, and sexual activity, and/or episodes of sadness or apathy; (c) the method results in improvement in the subject's depression over a defined period of time, as measured by one or more clinically-recognized depression rating scale, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%; and/or (d) each defined period of time is independently selected from about 1 day to about 10 days, about 10 days to about 30 days, about 30 days to about 3 months, about 3 months to about 6 months, about 6 months to about 12 months, and about greater than 12 months.

The one or more AD symptom can comprise cognitive impairment. In some embodiments, (a) progression or onset of the cognitive impairment is slowed, halted, or reversed over a defined period of time following administration of the composition, as measured by a medically-recognized technique; (b) the cognitive impairment is positively impacted by the administered composition, as measured by a medically-recognized technique; (c) the cognitive impairment is positively impacted by the administered composition, as measured by a medically-recognized technique and the positive impact on and/or progression of cognitive impairment is measured quantitatively or qualitatively by one or more techniques selected from Mini-Mental State Exam (MMSE), Mini-cog test, Montreal Cognitive Assessment (MoCA), and a computerized test selected from Cantab Mobile, Cognigram, Cognivue, Cognision, or Automated Neuropsychological Assessment Metrics; (d) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or (e) each defined period of time is independently selected from about 1 day to about 10 days, about 10 days to about 30 days, about 30 days to about 3 months, about 3 months to about 6 months, about 6 months to about 12 months, and about greater than 12 months.

The one or more AD symptom can comprise constipation. In some embodiments, (a) the composition causes the subject to have a bowel movement; and/or (b) the method results in an increase in the frequency of bowel movement in the subject; and/or (c) the method results in an increase in the frequency of bowel movement in the subject and the increase in the frequency of bowel movement is defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (d) as a result of the method the subject has the frequency of bowel movement recommended by a medical authority for the age group of the subject.

The one or more AD symptom can comprise neurodegeneration. In some embodiments, (a) the method results in treating, preventing, and/or delaying the progression and/or onset of neurodegeneration in the subject; (b) progression or onset of the neurodegeneration is slowed, halted, or reversed over a defined period of time following administration of the composition, as measured by a medically-recognized technique; (c) the neurodegeneration is positively impacted by the administered composition, as measured by a medically-recognized technique; (d) the progression of (b) and/or the positive impact of (c) is measured quantitatively or qualitatively by one or more techniques selected from electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; (e) the progression or onset of (b) is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or (f) each defined period of time is independently selected from about 1 day to about 10 days, about 10 days to about 30 days, about 30 days to about 3 months, about 3 months to about 6 months, about 6 months to about 12 months, and about greater than 12 months.

### Compositions and Administration

Provided herein are compositions comprising a CNS homing peptide. The composition can be a pharmaceutical composition comprising the CNS homing peptide and at least one pharmaceutically acceptable excipient. The pharmaceutical composition (or a pharmaceutically acceptable carrier, pharmaceutically acceptable salt, co-crystal, polymorph, hydrate, or solvate thereof) of the present disclosure can be for use in treating dementia (e.g., AD-related dementia). A kit can comprise any pharmaceutical composition disclosed herein and instructions for using the pharmaceutical composition to treat a disease. The composition can be administered to the subject by intravenous administration. The composition can be formulated for intravenous administration. The composition can be administered intravenously as a bolus, injection, infusion, or prolonged infusion The composition can be administered to the subject by subcutaneous injection, nasal administration, pulmonary administration, oral administration, parenteral administration, or nebulization. The composition can be administered to the subject once, twice, or three times a day. The composition can be administered to the subject daily, every two days, every three days, every week, every two weeks, or every month.

In some embodiments, the compositions disclosed herein do not comprise any additional therapeutic agent. The additional therapeutic agent can comprise:
(i) an acetylcholinesterase inhibitor selected from the group consisting of donepezil hydrochloride (ARICEPT, MEMAC), physostigmine salicylate (ANTILIRIUM), physostigmine sulfate (ESERINE), metrifonate, neostigmine, ganstigmine, pyridostigmine (MESTINON), ambenonium (MYTELASE), demarcarium, Debio 9902, rivastigmine (EXELON), ladostigil, NP-0361, galantamine hydrobromide (RAZADYNE, RIMINYL, NIVALIN), tacrine (COGNEX), tolserine, velnacrine maleate, memoquin, huperzine A (HUP-A), phenserine, edrophonium (ENLON, TENSILON), and INM-176;
(ii) an amyloid-β (or fragments thereof) selected from the group consisting of A1-15 conjugated to pan HLA DR-binding epitope (PADRE), ACC-001, ACI-01, ACI-24, AN-1792, Affitope AD-01, CAD106, and V-950;
(iii) an antibody to amyloid-β (or fragments thereof) selected from the group consisting of lecanemab (LEQEMBI), aducanumab (ADUHELM), ponezumab, solanezumab, bapineuzumab, AAB-002, ACI-01-Ab7, BAN-2401, intravenous Ig (GAMMAGARD), LY2062430 (humanized m266), R1450, ACU-5A5, and huC091;
(iv) an amyloid-lowering or -inhibiting agent (including those that reduce amyloid production, accumulation and fibrillization) selected from the group consisting of dimebon, davunetide, eprodisate, leuprolide, SK-PC-B70M, celecoxib, lovastatin, anapsos, oxiracetam, pramiracetam, varenicline, nicergoline, colostrinin, bisnorcymserine, NIC5-15 (Humanetics), E-2012 (Eisai), pioglitazone, clioquinol, PBT2 (Prana Biotechnology), flurbiprofen (ANSAID, FROBEN) and its R-enantiomertarenflurbil (FLURIZAN), nitroflurbiprofen, fenoprofen (FENOPRON, NALFON), ibuprofen (ADVIL, MOTRIN, NUROFEN), ibuprofen lysinate, meclofenamic acid, meclofenamate sodium (MECLOMEN), indomethacin (INDOCIN), diclofenac sodium (VOLTAREN), diclofenac potassium, sulindac (CLINORIL), sulindac sulfide, diflunisal (DOLOBID), naproxen (NAPROSYN), naproxen sodium (ANAPROX, ALEVE), ARC031, CAD-106 (Cytos), LY450139, insulin-degrading enzyme, the gingko biloba extract EGb-761 (ROKAN, TEBONIN), tramiprosate (CEREBRIL, ALZHEMED), eprodisate (FIBRILLEX, KIACTA), compound W [3,5-bis(4-nitrophenoxy)benzoic acid], NGX-96992, neprilysin, scyllo-inositol, atorvastatin (LIPITOR), simvastatin (ZOCOR), KLVFF-(EEX)3, SKF-74652, ibutamoren mesylate, BACE inhibitors such as ASP-1702, SCH-745966, JNJ-715754, AMG-0683, AZ-12304146, BMS-782450, GSK-188909, NB-533, E2609 and TTP-854; gamma secretase modulators; and RAGE (receptor for advanced glycation end-products) inhibitors, and PTI-777;
(v) an alpha-adrenergic receptor agonist selected from the group consisting of guanfacine (INTUNIV, TENEX), clonidine (CATAPRES), metaraminol (ARAMINE), methyldopa (ALDOMET, DOPAMET, NOVOMEDOPA), tizanidine (ZANAFLEX), phenylephrine, methoxamine, cirazoline, guanfacine (INTUNIV), lofexidine, xylazine, modafinil (PROVIGIL), adrafinil, and armodafinil (NUVIGIL);
(vi) a beta-adrenergic receptor blocking agent (beta blocker) selected from the group consisting of carteolol, esmolol (BREVIBLOC), labetalol (NORMODYNE, TRANDATE), oxprenolol (LARACOR, TRASACOR), pindolol (VISKEN), propanolol (INDERAL), sotalol (BETAPACE, SOTALEX, SOTACOR), timolol (BLOCADREN, TIMOPTIC), acebutolol (SECTRAL, PRENT), nadolol (CORGARD), metoprolol tartrate (LOPRESSOR), metoprolol succinate (TOPROL-XL), atenolol (TENORMIN), butoxamine, and SR 59230A;
(vii) an anticholinergic selected from the group consisting of amitriptyline (ELAVIL, ENDEP), butriptyline, benztropine mesylate (COGENTIN), trihexyphenidyl (ARTANE), diphenhydramine (BENADRYL), orphenadrine (NORFLEX), hyoscyamine, atropine (ATROPEN), scopolamine (TRANSDERM-SCOP), scopolamine methylbromide (PARMINE), dicycloverine (BENTYL, BYCLOMINE, DIBENT, DILOMINE), tolterodine (DETROL), oxybutynin (DITROPAN, LYRINEL XL, OXYTROL), penthienate bromide, propantheline (PRO-BANTHINE), cyclizine, imipramine hydrochloride (TOFRANIL), imipramine maleate (SURMONTIL), lofepramine, desipramine (NORPRAMIN), doxepin (SINEQUAN, ZONALON), trimipramine (SURMONTIL), and glycopyrrolate (ROBINUL);
(viii) an anticonvulsant selected from the group consisting of carbamazepine (TEGRETOL, CARBATROL), oxcarbazepine (TRILEPTAL), phenytoin sodium (PHENYTEK), fosphenytoin (CEREBYX, PRODILANTIN), divalproex sodium (DEPAKOTE), gabapentin (NEURONTIN), pregabalin (LYRICA), topirimate (TOPAMAX), valproic acid (DEPAKENE), valproate sodium (DEPACON), 1-benzyl-5-bromouracil, progabide, beclamide, zonisamide (TRERIEF, EXCEGRAN), CP-465022, retigabine, talampanel, and primidone (MYSOLINE);
(ix) an antipsychotic selected from the group consisting of lurasidone (LATUDA), aripiprazole (ABILIFY), chlorpromazine (THORAZINE), haloperidol (HALDOL), iloperidone (FANAPTA), flupentixol decanoate (DEPIXOL, FLUANXOL), reserpine (SERPLAN), pimozide (ORAP), fluphenazine decanoate, fluphenazine hydrochloride, prochlorperazine (COMPRO), asenapine (SAPHRIS), Ioxapine (LOXITANE), molindone (MOBAN), perphenazine, thioridazine, thiothixine, trifluoperazine (STELAZINE), ramelteon, clozapine (CLOZARIL), norclozapine (ACP-104), risperidone (RISPERDAL), paliperidone (INVEGA), melperone, olanzapine (ZYPREXA), quetiapine (SEROQUEL), talnetant, amisulpride, ziprasidone (GEODON), blonanserin (LONASEN), and ACP-103;
(x) a calcium channel blocker selected from the group consisting of omerizine, ziconotide, nilvadipine (ESCOR, NIVADIL), diperdipine, amlodipine (NORVASC, ISTIN, AMLODIN), felodipine (PLENDIL), nicardipine (CARDENE), nifedipine (ADALAT, PROCARDIA), MEM 1003 and its parent compound nimodipine (NIMOTOP), nisoldipine (SULAR), nitrendipine, lacidipine (LACIPIL, MOTENS), lercanidipine (ZANIDIP), lifarizine, diltiazem (CARDIZEM), verapamil (CALAN, VERELAN), AR-R 18565 (AstraZeneca), and enecadin;
(xi) a catechol O-methyltransferase (COMT) inhibitor selected from the group consisting of nitecapone, tolcapone (TASMAR), entacapone (COMTAN), and tropolone;
(xii) a central nervous system stimulant selected from the group consisting of atomoxetine, reboxetine, yohimbine, caffeine, phenmetrazine, phendimetrazine, pemoline, fencamfamine (GLUCOENERGAN, REACTIVAN), fenethylline (CAPTAGON), pipradol (MERETRAN), deanol, methylphenidate (DAYTRANA), methylphenidate hydrochloride (RITALIN), dexmethylphenidate (FOCALIN), amphetamine (alone or in combination with other CNS stimulants, e.g., ADDERALL (amphetamine aspartate, amphetamine sulfate, dextroamphetamine saccharate, and dextroamphetamine sulfate)), dextroamphetamine sulfate (DEXEDRINE, DEXTROSTAT), methamphetamine (DESOXYN), lisdexamfetamine (VYVANSE), and benzphetamine (DIDREX);
(xiii) a corticosteroid selected from the group consisting of prednisone (STERAPRED, DELTASONE), prednisolone (PRELONE), predisolone acetate (OMNIPRED, PRED MILD, PRED FORTE), prednisolone sodium phosphate (ORAPRED ODT), methylprednisolone (MEDROL), methylprednisolone acetate (DEPO-MEDROL), and methylprednisolone sodium succinate (A-METHAPRED, SOLU-MEDROL);
(xiv) a dopamine receptor agonist selected from the group consisting of apomorphine (APOKYN), bromocriptine (PARLODEL), cabergoline (DOSTINEX), dihydrexidine, dihydroergocryptine, fenoldopam (CORLOPAM), lisuride (DOPERGIN), terguride spergolide (PERMAX), piribedil (TRIVASTAL, TRASTAL), pramipexole (MIRAPEX), quinpirole, ropinirole (REQUIP), rotigotine (NEUPRO), SKF-82958, cariprazine, pardoprunox, and sarizotan;
(xv) a dopamine receptor antagonist selected from the group consisting of chlorpromazine, fluphenazine, haloperidol, loxapine, risperidone, thioridazine, thiothixene, trifluoperazine, tetrabenazine (NITOMAN, XENAZINE), 7-hydroxyamoxapine, droperidol (INAPSINE, DRIDOL, DROPLETAN), domperidone (MOTILIUM), L-741742, L-745870, raclopride, SB-277011A, SCH-23390, ecopipam, SKF-83566, and metoclopramide (REGLAN);
(xvi) a dopamine reuptake inhibitor selected from the group consisting of bupropion, safinamide, nomifensine maleate (MERITAL), vanoxerine and its decanoate ester DBL-583, and amineptine;
(xvii) a gamma-amino-butyric acid (GABA) receptor agonist selected from the group consisting of baclofen (LIORESAL, KEMSTRO), siclofen, pentobarbital (NEMBUTAL), progabide (GABRENE), and clomethiazole;
(xviii) a histamine 3 (H3) antagonist;
(xix) an immunomodulator selected from the group consisting of glatiramer acetate, MBP-8298 (synthetic myelin basic protein peptide), dimethyl fumarate, fingolimod, roquinimex (LINOMIDE), laquinimod, ABT-874 (human anti-IL-12 antibody; Abbott), rituximab (RITUXAN), alemtuzumab (CAMPATH), daclizumab (ZENAPAX), and natalizumab (TYSABRI);
(xx) an immunosuppressant selected from the group consisting of methotrexate (TREXALL, RHEUMATREX), mitoxantrone (NOVANTRONE), mycophenolate mofetil (CELLCEPT), mycophenolate sodium (MYFORTIC), azathioprine (AZASAN, IMURAN), mercaptopurine (PURI-NETHOL), cyclophosphamide (NEOSAR, CYTOXAN), chlorambucil (LEUKERAN), cladribine (LEUSTATIN, MYLINAX), alpha-fetoprotein, etanercept (ENBREL), and 4-(benzyloxy)-5-[(5-undecyl-2H-pyrrol-2-ylidene)methyl]-1H, 1′H-2,2′-bipyrrole;
(xxi) an interferon selected from the group consisting of interferon beta-la (AVONEX, REBIF) and interferon beta-1b (BETASERON, BETAFERON);
(xxii) a levodopa (or its methyl or ethyl ester), alone or in combination with a DOPA decarboxylase inhibitor (e.g., carbidopa (SINEMET, CARBILEV, PARCOPA), benserazide (MADOPAR), α-methyldopa, monofluromethyldopa, difluoromethyldopa, brocresine, or m-hydroxybenzylhydrazine);
(xxiii) a N-methyl-D-aspartate (NMDA) receptor antagonist selected from the group consisting of memantine (NAMENDA, AXURA, EBIXA), amantadine (SYMMETREL), acamprosate (CAMPRAL), besonprodil, ketamine (KETALAR), delucemine, dexanabinol, dexefaroxan, dextromethorphan, dextrorphan, traxoprodil, CP-283097, himantane, idantadol, ipenoxazone, L-701252, lancicemine, levorphanol (DROMORAN), LY-233536, LY-235959, methadone, (DOLOPHINE), neramexane, perzinfotel, phencyclidine, tianeptine (STABLON), dizocilpine, EAB-318, ibogaine, voacangine, tiletamine, riluzole (RILUTEK), aptiganel (CERESOTAT), gavestinel, and remacimide;
(xxiv) a monoamine oxidase (MAO) inhibitor selected from the group consisting of selegiline (EMSAM), selegiline hydrochloride (L-deprenyl, ELDEPRYL, ZELAPAR), dimethylselegilene, brofaromine, phenelzine (NARDIL), tranylcypromine (PARNATE), moclobemide (AURORIX, MANERIX), befloxatone, safinamide, isocarboxazid (MARPLAN), nialamide (NIAMID), rasagiline (AZILECT), iproniazide (MARSILID, IPROZID, IPRONID), CHF-3381 (Chiesi Farmaceutici), iproclozide, toloxatone (HUMORYL, PERENUM), bifemelane, desoxypeganine, harmine, harmaline, linezolid (ZYVOX, ZYVOXID), and pargyline (EUDATIN, SUPIRDYL);
(xxv) a muscarinic receptor agonist selected from the group consisting of cevimeline, levetiracetam, bethanechol chloride (DUVOID, URECHOLINE), itameline, pilocarpine (SALAGEN), NGX267, arecoline, L-687306, L-689660, furtrethonium iodide (FURAMON, FURANOL), furtrethonium benzensulfonate, furtrethonium p-toluenesulfonate, McN-A-343, oxotremorine, sabcomeline, AC-90222, and carbachol (CARBASTAT, MIOSTAT, CARBOPTIC);
(xxvi) a neuroprotective drug selected from the group consisting of bosutinib, condoliase, airmoclomol, lamotrigine, perampanel, aniracetam, minaprime, riluzole, N-hydroxy-1,2,4,9-tetrahydro-3H-carbazol-3-imine, desmoteplase, anatibant, astaxanthin, neuropeptide NAP, neurostrol, perampenel, ispronicline, bis(4-B-D-glucopyranosyloxybenzyl)-2-β-D-glucopyranosyl-2-isobutyltartrate (also known as dactylorhin B or DHB), formobactin, xaliproden (XAPRILA), lactacystin, dimeboline hydrochloride (DIMEBON), disufenton (CEROVIVE), arundic acid (ONO-2506, PROGLIA, CEREACT), citicoline, edaravone (RADICUT), AEOL-10113, AEOL-10150, AGY-94806, granulocyte-colony stimulating factor, BAY-38-7271, ancrod (VIPRINEX, ARWIN), DP-b99, HF-0220 (17-ß-hydroxyepiandrosterone; Newron Pharmaceuticals), HF-0420 (also known as oligotropin), pyridoxal 5′-phosphate, microplasmin, S-18986, piclozotan, NPO31112, tacrolimus, L-seryl-L-methionyl-L-alanyl-L-lysyl-L-glutamyl-glycyl-L-valine, AC-184897, ADNF-14, stilbazulenyl nitrone, SUN-N8075, and zonampanel;
(xxvii) a nicotinic receptor agonist selected from the group consisting of epibatidine, bupropion, CP-601927, varenicline, ABT-089, ABT-594, AZD-0328, EVP-6124, R3487, TC-4959 and TC-5619 (both Targacept), and RJR-2403;
(xxviii) a norepinephrine (noradrenaline) reuptake inhibitor selected from the group consisting of atomoxetine (STRATTERA), doxepin (APONAL, ADAPIN, SINEQUAN), nortriptyline (AVENTYL, PAMELOR, NORTRILEN), amoxapine (ASENDIN, DEMOLOX, MOXIDIL), reboxetine (EDRONAX, VESTRA), viloxazine (VIVALAN), maprotiline (DEPRILEPT, LUDIOMIL, PSYMION), bupropion (WELLBUTRIN), and radaxafine;
(xxix) a phosphodiesterase (PDE) inhibitor;
(xxx) a quinoline selected from the group consisting of quinine (including its hydrochloride, dihydrochloride, sulfate, bisulfate and gluconate salts), chloroquine, sontoquine, hydroxychloroquine (PLAQUENIL), mefloquine (LARIAM), and amodiaquine (CAMOQUIN, FLAVOQUINE);
(xxxi) a β-secretase inhibitor selected from the group consisting of ASP-1702, SCH-745966, JNJ-715754, AMG-0683, AZ-12304146, BMS-782450, GSK-188909, NB-533, LY-2886721, E-2609, HPP-854, (+)-phenserine tartrate (POSIPHEN), LSN-2434074, KMI-574, SCH-745966, Ac-rER (N2-acetyl-D-arginyl-L-arginine), Ioxistatin, and CA074Me;
(xxxii) a y-secretase inhibitor and/or modulator selected from the group consisting of BMS-708163 (Avagacest), DSP8658 (Dainippon), ITI-009, L-685458, ELAN-G, ELAN-Z, 4-chloro-N-[(2S)-3-ethyl-1-hydroxypentan-2-yl]benzenesulfonamide;
(xxxiii) a serotonin (5-hydroxytryptamine) 1A (5-HT1A) receptor antagonist selected from the group consisting of spiperone, levo-pindolol, BMY 7378, NAD-299, S-(−)-UH-301, NAN 190, lecozotan;
(xxxiv) a serotonin (5-hydroxytryptamine) 2C (5-HT2c) receptor agonist selected from the group consisting of vabicaserin and zicronapine;
(xxxv) a serotonin (5-hydroxytryptamine) 4 (5-HT4) receptor agonist;
(xxxvi) a serotonin (5-hydroxytryptamine) 6 (5-HT6) receptor antagonist selected from the group consisting of A-964324, AVI-101, AVN-211, mianserin (TORVOL, BOLVIDON, NORVAL), methiothepin, ritanserin, ALX-1161, ALX-1175, MS-245, LY-483518, MS-245, Ro 04-6790, Ro 43-68544, Ro 63-0563, Ro 65-7199, Ro 65-7674, SB-399885, SB-214111, SB-258510, SB-271046, SB-357134, SB-699929, SB-271046, SB-742457, Lu AE58054, and PRX-07034 (Epix);
(xxxvii) a serotonin (5-HT) reuptake inhibitor selected from the group consisting of alaproclate, citalopram (CELEXA, CIPRAMIL), escitalopram (LEXAPRO, CIPRALEX), clomipramine (ANAFRANIL), duloxetine (CYMBALTA), femoxetine (MALEXIL), fenfluramine (PONDIMIN), norfenfluramine, fluoxetine (PROZAC), fluvoxamine (LUVOX), indalpine, milnacipran (IXEL), paroxetine (PAXIL, SEROXAT), sertraline (ZOLOFT, LUSTRAL), trazodone (DESYREL, MOLIPAXIN), venlafaxine (EFFEXOR), zimelidine (NORMUD, ZELMID), bicifadine, desvenlafaxine (PRISTIQ), brasofensine, vilazodone, cariprazine, neuralstem, and tesofensine;
(xxxviii) a trophic factor selected from the group consisting of nerve growth factor (NGF), basic fibroblast growth factor (bFGF; ERSOFERMIN), neurotrophin-3 (NT-3), cardiotrophin-1, brain-derived neurotrophic factor (BDNF), neublastin, meteorin, and glial-derived neurotrophic factor (GDNF), and agents that stimulate production of trophic factors, such as propentofylline, idebenone, PYM50028 (COGANE; Phytopharm), and AIT-082 (NEOTROFIN);
(xxxix) a Glycine transporter-1 inhibitor selected from paliflutine, ORG-25935, JNJ-17305600, and ORG-26041;
(xl) an AMPA-type glutamate receptor modulator selected from the group consisting of perampanel, mibampator, selurampanel, GSK-729327, N-{(3S,4S)-4-[4-(5-cyanothiophen-2-yl)phenoxy]tetrahydro-furan-3-yl}propane-2-sulfonamide;
(xli) a Janus kinase inhibitor (JAK) selected from tofacitinib, ruxolitinib, baricitinib, CYT387, GLPG0634, lestaurtinib, pacritinib, and TG101348; and
(xlii) an Interleukin-1 receptor-associated kinase 4 inhibitor (IRAK4).

In some embodiments, the therapeutic agent comprises an non-steroidal anti-inflammatory drug (NSAID). In some embodiments, the composition does not comprise an NSAID. In some embodiments, the CNS homing peptide is not associated with any additional therapeutic agent (e.g., an NSAID). The NSAID can comprise aspirin, diflunisal, salicylic acid, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, aceclofenac, bromfenac, nabumetone, piroxicam, meloxicam, tenoxicam, droxicam, lornoxicam, isoxicam, phenylbutazone (bute), mefenamic acid, meclofenamic acid, flufenamic acid, tolfenamic acid, celecoxib, rofecoxib, valdecoxib, parecoxib, lumiracoxib, etoricoxib, firocoxib, nimesulide, clonixin, licofelone, H-harpagide, or a combination thereof.

The pharmaceutical composition can comprise different numbers of CNS homing peptides in different embodiments. In some embodiments, the pharmaceutical composition comprises, comprises about, comprises at least, comprises at least about, comprises at most, or comprises at most about, 1, 2, 3, 4, 5, 6, 7, 8, 9, 10, 11, 12, 13, 14, 15, 16, 17, 18, 19, 20, 30, 40, 50, 60, 70, 80, 90, 100, or a number or a range between any two of these values, CNS homing peptides. Two or more of the CNS homing peptides can be co-administered simultaneously or sequentially. The therapeutically effective amount of the pharmaceutical composition can be different in different embodiments or different time periods or cycles. For example, the therapeutically effective amount of the pharmaceutical composition of the first cycle and the therapeutically effective amount of subsequent cycles can be different. In some embodiments, the therapeutically effective amount of the pharmaceutical composition is, is about, is at least, is at least about, is at most, or is at most about, 10 μg, 20 μg, 30 μg, 40 μg, 50 μg, 60 μg, 70 μg, 80 μg, 90 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 550 mg, 600 mg, 650 mg, 700 mg, 750 mg, 800 mg, 850 mg, 900 mg, 950 mg, 1000 mg, 1 g, 2 g, 3 g, 4 g, 5 g, 6 g, 7 g, 8 g, 9 g, 10 g, or a number or a range between any two of these values. For example, the therapeutically effective amount of the pharmaceutical composition is about 1 mg to about 100 mg of the pharmaceutical composition.

The number of cycle(s) can be different in different embodiments. In some embodiments, the number of cycle(s) is, is about, is at least, is at least about, is at most, or is at most about, 1, 2, 3, 4, 5, 6, 7, 8, 9, 10, 11, 12, 13, 14, 15, 16, 17, 18, 19, 20, 30, 40, 50, 60, 70, 80, 90, 100, or a number or a range between any two of these values. The time period or the length of a cycle can be different in different embodiments. In some embodiments, the time period or the length of a cycle is, is about, is at least, is at least about, is at most, or is at most about, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 2 weeks, 3 weeks, 4 weeks, 5 weeks, 1 month, 2 months, 3 months, 4 months, 5 months, 6 months, 7 months, 8 months, 9 months, 10 months, 11 months, 12 months, or a number or a range between any two of these values.

The number of dose(s) administered per time period or per cycle, can be different in different embodiments. In some embodiments, the number of dose(s) administered per time period or per cycle is, is about, is at least, is at least about, is at most, or is at most about, 1, 2, 3, 4, 5, 6, 7, 8, 9, 10, 11, 12, 13, 14, 15, 16, 17, 18, 19, 20, 30, 40, 50, 60, 70, 80, 90, 100, or a number or a range between any two of these values. The total number of dose(s) administered can be different in different embodiments. In some embodiments, the total number of dose(s) administered is, is about, is at least, is at least about, is at most, or is at most about, 1, 2, 3, 4, 5, 6, 7, 8, 9, 10, 11, 12, 13, 14, 15, 16, 17, 18, 19, 20, 30, 40, 50, 60, 70, 80, 90, 100, or a number or a range between any two of these values.

The therapeutically effective amount of the pharmaceutical composition can be different in different embodiments or different time periods or cycles. For example, the therapeutically effective amount comprises about 10 μg to about 3000 μg of the pharmaceutical composition per kilogram of the body weight of the subject (μg/kg) being administered the pharmaceutical composition. In some embodiments, the therapeutically effective amount comprises, comprises about, comprises at least, comprises at least about, comprises at most, or comprises at most about, 100 ng/kg, 200 ng/kg, 300 ng/kg, 400 ng/kg, 500 ng/kg, 600 ng/kg, 700 ng/kg, 800 ng/kg, 900 ng/kg, 1000 ng/kg, 2 μg/kg, 3 μg/kg, 4 μg/kg, 5 μg/kg, 6 μg/kg, 7 μg/kg, 8 μg/kg, 9 μg/kg, 10 μg/kg, 20 μg/kg, 30 μg/kg, 40 μg/kg, 50 μg/kg, 60 μg/kg, 70 μg/kg, 80 μg/kg, 90 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 6 mg/kg, 7 mg/kg, 8 mg/kg, 9 mg/kg, 10 mg/kg, or a number or a range between any two of these values, of the pharmaceutical composition.

Some embodiments provided herein are directed to an effective amount of a pharmaceutical composition comprising CNS homing peptide and at least one pharmaceutically acceptable carrier or excipient. Some embodiments provided herein are directed to a pharmaceutical composition comprising an effective amount of a CNS homing peptide and at least one pharmaceutically acceptable carrier or excipient.

Carriers or excipients can be used to produce compositions. The carriers or excipients can be chosen to facilitate administration of the compound or composition. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

Suitable dosage forms, in part, depend upon the use or the route of administration, for example, oral, transdermal, transmucosal, inhalant, or by injection (parenteral). Such dosage forms should allow the compound to reach target cells. Other factors include considerations such as toxicity and dosage forms that retard the compound or composition from exerting its effects.

The compound or composition can be administered by different routes including intravenous, intraperitoneal, subcutaneous, intramuscular, oral, transmucosal, rectal, transdermal, or inhalant. In some embodiments, the composition can be administered by oral administration. For oral administration, for example, the compound or composition can be formulated into conventional oral dosage forms such as capsules, tablets, and liquid preparations such as syrups, elixirs, and concentrated drops.

Administration can also be by transmucosal, topical, transdermal, or inhalant means. For transmucosal, topical or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal).

The topical compositions of this disclosure are formulated as oils, creams, lotions, ointments, and the like by choice of appropriate carriers known in the art. Suitable carriers include vegetable or mineral oils, white petrolatum (white soft paraffin), branched chain fats or oils, animal fats and high molecular weight alcohol (greater than C12). In another embodiment, the carriers are those in which the active ingredient is soluble. Emulsifiers, stabilizers, humectants and antioxidants may also be included as well as agents imparting color or fragrance, if desired. Creams for topical application are formulated from a mixture of mineral oil, self-emulsifying beeswax and water in which mixture the active ingredient, dissolved in a small amount solvent (e.g. an oil), is admixed. Additionally, administration by transdermal means may comprise a transdermal patch or dressing such as a bandage impregnated with an active ingredient and optionally one or more carriers or diluents known in the art. To be administered in the form of a transdermal delivery system, the dosage administration will, of course, be continuous rather than intermittent throughout the dosage regimen.

Injection (parenteral administration) may be used, e.g., intramuscular, intravenous, intraperitoneal, and/or subcutaneous. For injection, the compound or composition can be formulated in sterile liquid solutions, such as in physiologically compatible buffers or solutions, such as saline solution, Hank's solution, or Ringer's solution. In addition, the compound or composition may be formulated in solid form and redissolved or suspended immediately prior to use. Lyophilized forms can also be produced. In some embodiments, the composition is administered to the subject by intravenous administration. In some embodiments, the composition is formulated for intravenous administration. In some embodiments, the composition is administered intravenously as a bolus, injection, infusion, or prolonged infusion.

Disclosed herein include compositions. In some embodiments, the composition comprises: a central nervous system (CNS) homing peptide comprising an amino acid sequence of any one of SEQ ID NOs: 1-22. In some embodiments, the composition does not comprise any additional therapeutic agent.

Disclosed herein include compositions for use. In some embodiments, the composition comprises: a CNS homing peptide comprising an amino acid sequence of any one of SEQ ID NOs: 1-22 for use in treating AD. In some embodiments, the composition comprises: a CNS homing peptide comprising an amino acid sequence of any one of SEQ ID NOs: 1-22 for use in delaying or reducing the likelihood of onset of AD. In some embodiments, the composition comprises: a CNS homing peptide comprising an amino acid sequence of any one of SEQ ID NOs: 1-22 for use in treating, preventing, or reversing cognitive decline in clinical or pre-clinical AD. In some embodiments, the composition comprises: a CNS homing peptide comprising an amino acid sequence of any one of SEQ ID NOs: 1-22 for use in treating, delaying or reversing dementia. In some embodiments, the composition comprises: a CNS homing peptide comprising an amino acid sequence of any one of SEQ ID NOs: 1-22 for use in delaying or reversing the progression of AD. In some embodiments, the compositions disclosed herein do not comprise any additional therapeutic agent. The composition can be a pharmaceutical composition comprising the CNS homing peptide and at least one pharmaceutically acceptable excipient. The composition can be formulated for oral administration, intravenous administration, subcutaneous administration, or a combination thereof.

In some embodiments, the present disclosure provides kits that include a therapeutic agent (e.g., a CNS homing peptide) or a pharmaceutical composition thereof. In some embodiments, the peptide or composition is packaged, e.g., in a vial, bottle, flask, which may be further packaged, e.g., within a box, envelope, or bag; the compound or composition is approved by the U.S. Food and Drug Administration or similar regulatory agency for administration to a mammal, e.g., a human; the compound or composition is approved for administration to a mammal, e.g., a human, for a CNS disease or condition; the kits described herein may include written instructions for use and/or other indication that the compound or composition is suitable or approved for administration to a mammal, e.g., a human, for a disease or condition as described herein; and the compound or composition may be packaged in unit dose or single dose form, e.g., single dose pills, capsules, or the like.

The kit can comprise: at least one of a CNS homing peptide comprising an amino acid sequence of any one of SEQ ID NOs: 1-22; and a label indicating at least one of: (a) the kit is for preventing or delaying the onset of AD, (b) the kit is for treating AD, (c) the kit is for treating, preventing, or reversing cognitive decline in clinical or pre-clinical AD, (d) the kit is for delaying or reversing the progression of AD, and (e) the kit is for delaying, preventing, or reversing dementia. The dementia can be AD-related dementia. The dementia can be vascular dementia, Lewy body dementia, fronto-temporal dementia, or mixed dementia.

In some embodiments, kit does not comprise any additional therapeutic agent for any of: (a) preventing or delaying the onset of AD, (b) treating AD, (c) treating, preventing, or reversing cognitive decline in clinical or pre-clinical AD, (d) delaying or reversing the progression of AD; and (e) delaying, preventing, or reversing dementia. The dementia can be AD-related dementia. The dementia can be vascular dementia, Lewy body dementia, fronto-temporal dementia, or mixed dementia. The AD can be mild cognitive impairment associated with AD, early stage AD, mid-stage AD, and/or late-stage AD. The AD can be sporadic AD or familial AD.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of treating, preventing, or reversing dementia in a subject in need thereof, comprising administering to the subject a therapeutically effective amount of a composition comprising a central nervous system (CNS) homing peptide, thereby treating, preventing, or reversing dementia in the subject; and wherein the composition does not comprise any additional therapeutic agent.

2. The method of claim 1, wherein the dementia is Alzheimer's disease (AD)-related dementia, vascular dementia, Lewy body dementia, fronto-temporal dementia, or mixed dementia.

3. (canceled)

4. (canceled)

5. A method of treating Alzheimer's disease (AD), or delaying or reducing the likelihood of onset of AD, comprising administering to a subject in need thereof a therapeutically effective amount of a composition comprising a central nervous system (CNS) homing peptide, thereby treating AD or delaying or reducing the likelihood of onset of AD in the subject; and wherein the composition does not comprise any additional therapeutic agent.

6. (canceled)

7. (canceled)

8. The method of claim 5, wherein the Alzheimer's disease (AD) is clinical or pre-clinical Alzheimer's disease (AD), and wherein cognitive decline in clinical or pre-clinical AD in the subject is treated, prevented or reversed.

9. (canceled)

10. (canceled)

11. The method of claim 5, wherein the progression of Alzheimer's disease (AD) is delayed or reversed.

12. (canceled)

13. (canceled)

14. The method of claim 1, wherein the CNS homing peptide comprises an amino acid sequence of any one of SEQ ID NOs: 1-22.

15. The method of claim 1, wherein the CNS homing peptide is capable of homing to the entorhinal cortex, the cerebral cortex, the hippocampus, or any combination thereof.

16.-18. (canceled)

19. The method of claim 1, comprising identifying the subject in need thereof, wherein the subject in need thereof is a subject at a risk of having Alzheimer's disease (AD), a subject having AD, or a subject suspected of having AD.

20.-23. (canceled)

24. The method of claim 1, wherein the subject in need thereof has at least one mutation in one or more genes associated with Alzheimer's disease, wherein the one or more genes comprise apolipoprotein E (APOE), amyloid precursor protein (APP), presenilin 1 (PSEN1), and/or presenilin 2 (PSEN2).

25.-27. (canceled)

28. The method of claim 1, wherein the composition is administered to the subject by intravenous administration, subcutaneous injection, nasal administration, pulmonary administration, oral administration, parenteral administration, or nebulization.

29. (canceled)

30. The method of claim 28, wherein the composition is administered intravenously as a bolus, injection, infusion, or prolonged infusion.

31.-34. (canceled)

35. The method of claim 1, wherein administering the composition reduces formation of plaques, amyloid fibril formation, amyloid-induced cellular toxicity or microglial activation, amyloid-induced neurotoxicity, and/or the rate or amount of amyloid aggregation, fibril formation, or deposition, lessens the degree of amyloid deposition, reduces amyloid-induced inflammation, results in reduction of neuroinflammation, or a combination thereof, reduces or slows down the formation of tangles containing hyperphosphorylated tau and/or reduces the concentration or the amount of phosphorylated tau in the brain of the subject.

36.-42. (canceled)

43. The method of claim 1, wherein the progression or onset of AD is measured quantitatively or qualitatively by at least one technique selected from the group consisting of electroencephalogram (EEG), neuroimaging, functional MRI, structural MRI, diffusion tensor imaging (DTI), [18F]fluorodeoxyglucose (FDG) PET, agents that label amyloid beta or tau, [18F]F-dopa PET, radiotracer imaging, volumetric analysis of regional tissue loss, specific imaging markers of abnormal protein deposition, multimodal imaging, and biomarker analysis (plasma or cerebrospinal fluid).

44. (canceled)

45. The method of claim 1, wherein administering the composition treats or prevents at least one AD symptom.

46. The method of claim 1, wherein administering the composition treats or prevents at least one AD symptom by at least about 10%.

47. The method of claim 1, wherein the progression or onset of AD is slowed or reversed by at least about 5%.

48. The method of claim 45, wherein the AD symptom is selected from the group consisting of:
(a) a symptom from the Integrated Alzheimer's Disease Rating Scale (iADRS) selected from the group consisting of personal belonging management, selection of clothes, ability to dress self, ability to clean habitation, financial management ability, writing ability, ability to keep appointments, ability to use telephone, ability to prepare food for self, travel ability, awareness of current events, reading ability, interest in television, ability to shop for self, ability to remain alone, ability to perform chores, ability to perform a hobby or game, driving ability, self-management of medications, ability to initiate and finish complex tasks, and ability to initiate and finish simple tasks;
(b) a symptom from the Alzheimer's Disease Assessment Scale-Cognitive subscale (ADAS-Cog) selected from the group consisting of learning, naming, command following, ideational praxis, constructional praxis, orientation, and recognition memory;
(c) a symptom from the Alzheimer's Disease Cooperative Study-instrumental Activities of Daily Living (ADCS-iADL) wherein the symptom is any of the symptoms recited in (a) or (b);
(d) constipation;
(e) depression;
(f) cognitive impairment;
(g) short term memory impairment;
(h) long term memory impairment;
(i) concentration impairment;
(j) coordination impairment;
(k) mobility impairment;
(l) speech impairment;
(m) mental confusion;
(n) sleep problem, sleep disorder, or sleep disturbance;
(o) circadian rhythm dysfunction;
(p) REM disturbed sleep;
(q) REM behavior disorder;
(r) hallucinations;
(s) fatigue;
(t) apathy;
(u) erectile dysfunction;
(v) mood swings;
(w) urinary incontinence;
(x) mild cognitive impairment; and
(y) neurodegeneration.

49.-54. (canceled)

55. A composition comprising a central nervous system (CNS) homing peptide comprising an amino acid sequence of any one of SEQ ID NOs: 1-22, wherein the composition does not comprise any additional therapeutic agent.

56.-62. (canceled)

63. A kit, comprising
the composition of claim 5; and
a label indicating at least one of:
(a) the kit is for preventing or delaying the onset of Alzheimer's disease (AD),
(b) the kit is for treating AD,
(c) the kit is for treating, preventing, or reversing cognitive decline in clinical or pre-clinical AD,
(d) the kit is for delaying or reversing the progression of AD, and
(e) the kit is for delaying, preventing, or reversing dementia.

64. The kit of claim 63, wherein the kit does not comprise any additional therapeutic agent for any of:
(a) preventing or delaying the onset of AD,
(b) treating AD,
(c) treating, preventing, or reversing cognitive decline in clinical or pre-clinical Alzheimer's disease (AD),
(d) delaying or reversing the progression of AD; and
(e) delaying, preventing, or reversing dementia.

65. (canceled)

66. (canceled)
